(19) United States
(12) Patent Application Publication
(10) Pub. No.: US 2024/0050396 A1
(43) Pub. Date: Feb. 15, 2024

(54) COMPOSITIONS AND METHODS FOR THE TREATMENT OF SOLID TUMORS

(71) Applicant: POLYPID LTD., Petach Tikva (IL)

(72) Inventor: Noam EMANUEL, Petach Tikva (IL)

(21) Appl. No.: 18/266,776

(22) PCT Filed: Dec. 21, 2021
(86) PCT No.: PCT/IB2021/062116

§ 371 Date: Jun. 12, 2023

Related U.S. Application Data
(60) Provisional application No. 63/128,218, filed on Dec. 21, 2020.
(60) Provisional application No. 63/231,662, filed on Aug. 10, 2021.
(60) Provisional application No. 63/243,147, filed on Sep. 12, 2021.

(51) Int. Cl. A61K 31/337; A61K 9/16; A61K 31/495; A61P 35/00

(52) U.S. Cl. CPC A61K 31/337; A61K 9/1617; A61K 9/1611; A61K 9/1647; A61K 31/495; A61P 35/00

(57) ABSTRACT

The present invention provides methods for treating solid tumors, reducing local tumor recurrence and tumor metastatic spreading, by administering directly into the tumor or to the tumor resection cavity a pharmaceutical composition comprising a particulate biodegradable substrate coated with a polymer-lipid based matrix which provide local sustained release of a taxane chemotherapeutic drug. The present invention further provides methods for treating chemotherapy resistant tumors.

This application claims the benefit of U.S. provisional application No. 63/128,218, filed Dec. 21, 2020, U.S. provisional application No. 63/231,662, filed Aug. 10, 2021 and U.S. provisional application No. 63/243,147, filed Sep. 12, 2021, the entirety of which are incorporated herein by reference.

### FIELD OF THE INVENTION

The present invention generally relates to sustained release compositions of chemotherapeutic agents and uses thereof for the local treatment of solid tumors, the prevention of post-resection cancer recurrence and metastasis.

### BACKGROUND

Systemic therapies often fail due to difficulty in achieving therapeutic levels of the drug in the tumor and its' surroundings for a sufficient duration to effectively kill malignant tumors. Dose escalation could address this issue, but the trade-off between efficacy, incremental toxicity and associated costs remains controversial.

The fundamental limitations of systemic chemotherapeutics administration have prompted the development of local drug delivery platforms as a solution to increase effectiveness and reduce side effects.

Local drug delivery provides several advantages to systemic drug administration such as oral or intravenous dosing, that make them promising therapeutics for cancer. Drug-eluting depots are capable of providing high concentrations of drugs locally at disease sites, while lowering systemic peaks in drug presentation via sustained drug release. Furthermore, local sustained drug delivery systems provide continuous drug presence, improving disease outcomes and patient compliance. Yet further, local drug delivery reduces and even prevents systemic side effects often seen with systemic drug dosing. These advantages make depots particularly promising in cancer therapy for the prevention of tumor recurrence and metastasis particularly at dirty surgical margins following surgical resection where sustained drug presentation can affect cancer cells left around the surgical incision with minimal or no appreciable systemic side effects. Various technologies are actively pursued for local delivery including polymeric biodegradable sustained release systems in the form of micro- or nanoparticles and implantable films or patches which typically suffer from burst and decaying release profiles. One clinically approved therapy, Gliadel®, uses a polyanhydride carrier (polifeposan) which affords sustained release of carmustine into the extracellular fluids of the brain, eliminating the need for the drug to cross the blood-brain barrier. One of the limitations of depot technologies based on biodegradable polyesters and polyanhydrides is the relatively short period of drug release available with many systems and the potential for toxicity due to dose dumping (burst effect) and inconsistent drug release. Gliadel®, for example, releases most of the drug within 5-10 days and demonstrates a burst release in the first 12 h (Brudno et al, Biomaterials 178 (2018) 373-382). Because the initial burst release translates to excessive local or systemic drug concentrations, the burst effect further limits the total amount of drug that can be loaded into the depots. Another important limitation is the low penetration of the released drug into the brain tissue. The drug penetration using Gliadel®, only extends to a maximum distance of 5 mm away from the resected tumor, and only for a short period of 1-2 days post-surgery (Dan Bunis et al. Efficacy of nanoparticle-encapsulated BCNU delivery in apCPP:SA scaffold for treatment of Glioblastoma Multiforme, 2012). U.S. Pat. No. 9,956,172 discloses drug delivery multilayered implants or wafers for positioning adjacent to biological tissues for delivering drugs thereto, particularly, for delivering chemotherapeutic drugs to the brain after the resection of brain tumor. The implants disclosed in U.S. Pat. No. 9,956,172 comprise a drug containing layer comprising the drug, a lipid and a hydrophilic polymer or a pore forming agent and a hydrophobic coating comprising a hydrophobic agent.

Glioblastoma multiforme (GBM) is one of the most common and aggressive forms of brain tumor, accounting for 50-60% of all brain cancers in human and is associated with low median survival rate. GBM is generally characterized by high lethality, invasiveness, excessive growth, and a poor prognosis. The current standard treatment for patients suffering from brain tumors comprised of tumor resection surgery followed by chemotherapy (typically oral temozolomide) and radiation treatments both given about a month after surgery. This delayed treatment allows the wound to begin the healing process. However, difficulties in surgical excision, and the severe adverse effects associated with irradiation and chemotherapy, hinder these approaches. On top of that the disadvantage of the delay is that cancer cells continue to grow during this period.

Docetaxel is an anti-mitotic taxane drug, considered to be one of the most effective drugs against brain tumors, typically given systemically by iv infusion. However, its high molecular weight and lipophilicity results limit its activity against brain tumor mainly due to limited transport across the blood brain barrier and poor penetration of the blood brain tumor barrier. Decetaxel is known for causing severe adverse events including infections, neutropenia, hypersensitivity, thrombocytopenia, neuropathy and many more.

International Publication No. WO 2010/007623 to one of the inventors of the present invention and others, the contents of which are incorporated herein by reference, discloses drug delivery compositions for controlled release of an active ingredient, comprising a lipid-based matrix with a biodegradable polymer. These drug delivery compositions enable to entrap a large variety of one or more biologically active molecules and to release them at a pre-programmed rate for periods ranging from several days to several months.

There is a need for the development of localized safe and robust anti-cancer treatments with taxanes in general and docetaxel in particular with reduced systemic toxicity, capable of enriching its payload concentration at tumor sites, present increased penetration to the target tumor cells and which will promote the eradication of tumor cells and at the time reduce the chances of the tumor acquiring resistance and overcome drug resistance mechanisms.

### SUMMARY OF THE INVENTION

The present invention provides sustained release anti-neoplastic compositions, as well as methods which utilize such compositions for the local treatment of cancer, prevention of cancer recurrence and inhibition of tumor metastasis.

Within a first aspect of the present invention, methods for treating solid tumors are provided comprising administering to a subject with a solid tumor a pharmaceutical composition comprising a particulate biodegradable substrate coated with a polymer-lipid-based matrix comprising a taxene. Following its application to the tumor site, the pharmaceutical composition provides local controlled release of the taxene drug at the tumor site and its surrounding over a predetermined, prolonged period of time, preferably up to 10 weeks, thereby improving the therapeutic effect of the drug. According to some embodiments, the pharmaceutical composition is administered to a site of tumor excision after the resection of the tumor, thereby killing the remaining cancer cells at the tumor excision cavity or in close proximity to the resected tissue and inhibiting the local recurrence of cancer. According to some embodiments, the solid tumor is at least one of brain tumor, colon carcinoma, prostate cancer, lung cancer, pancreatic cancer, breast cancer, esophageal cancer, gastric cancer, head & neck cancer and soft tissue sarcomas. According to certain embodiments the solid tumor is a brain tumor selected from a glioblastoma or glioblastoma multiforme, a high-grade intrinsic brain tumor and metastases of another tumor in the brain. According to specific embodiments, the brain tumor is glioblastoma multiforme.

Within a second aspect of the present invention, local sustained release compositions are provided comprising a particulate biodegradable substrate coated or impregnated with a polymer-lipid-based matrix comprising a taxene embedded therewithin, said composition stabilizing the taxene and slowing down the taxane's transition into its' 7-epimeric impurities during storage and further during its' extended-release period.

The present invention is based in part on experimental results showing that a single application of a sustained release composition comprising docetaxel, according to some embodiments of the invention, at the intra-operative setting post-tumor partial resection in a syngeneic mouse model for solid tumors of colon carcinoma resistant to docetaxel resulted in 75% overall tumor free survival at the end of the study (day 39 post surgery) compared to only 25% overall tumor free survival in a group treated with five cycles of systemic docetaxel treatment and no-survival in the untreated arm. Additionally, mice treated with said compositions showed 25% overall tumor recurrence at the end of the study as compared to 75% recurrence in the extensive systemic treatment and 100% recurrence in the untreated arm. Moreover, the arm treated with the docetaxel sustained release composition displayed delayed tumor recurrence 30 days after tumor resection, compared to a delayed tumor recurrence of only 9 days in both the systemic treatment arm and the non-treated control arm as determine by the first tumor related mortality in each group.

Furthermore, docetaxel sustained release composition according to certain embodiments of the invention induced strong inhibition of tumor growth and recurrence in a partially resected human glioblastoma subcutaneous mouse model. A single local application of said composition induced 98% tumor growth inhibition (day 41 post operation) compared to the untreated control (p<0.001), and 66% tumor growth inhibition compared to multiple injections of systemic chemotherapy treatment arm (p=0.0165). The day 41 survival rate for the docetaxel sustained release composition was much higher than for the systemic treated mice or for the untreated mice with 60%, 20%, and 10% survival, respectively.

Yet further, the docetaxel composition, applied locally next to the non-resected glioblastoma brain tumor in a rat model, showed a 40% survival rate at day 23 following the beginning of treatment, as compared to a 0% survival rate in the standard systemic treatment arm (Temozolomide 33.5 mg/kg, 5 treatment days), the placebo arm (composition without Docetaxel) and in the untreated control arm.

According to some embodiments of the invention, the method for treating a solid tumor comprises administering to a subject with a solid tumor a pharmaceutical composition comprising: (a) a particulate biodegradable substrate; (b) a biodegradable polymer; (c) at least one phospholipid having hydrocarbon chains of at least 12 carbons and (d) a taxene. According to some embodiments, the pharmaceutical composition further comprises a sterol. According to various embodiments the taxene is selected from the group consisting of docetaxel, paclitaxel, derivatives of paclitaxel and cabazitaxel. According to specific embodiments the taxene is docetaxel. According to some embodiments, the solid tumor is at least one of brain tumor, prostate cancer, lung cancer, pancreatic cancer, breast cancer, esophageal cancer, gastric cancer, head & neck cancer and soft tissue sarcomas. According to certain embodiments the solid tumor is a brain tumor selected from a glioblastoma or glioblastoma multiforme and a high-grade intrinsic brain tumor. According to specific embodiments, the brain tumor is glioblastoma multiforme. According to some embodiments, the tumor is a chemotherapy resistant tumor. According to some embodiments, the tumor is a taxane resistant tumor.

According to some embodiments of the invention, the present invention provides a method for reducing tumor cell regrowth at a site of solid tumor excision, comprising the administration to the site of solid tumor excision a pharmaceutical composition comprising: (a) a particulate biodegradable substrate; (b) a biodegradable polymer; (c) at least one phospholipid having hydrocarbon chains of at least 12 carbons and (d) a taxene. According to some embodiments, the pharmaceutical composition further comprises a sterol. According to various embodiments the taxene is selected from the group consisting of docetaxel, paclitaxel, derivatives of paclitaxel and cabazitaxel. According to specific embodiments the taxene is docetaxel. According to some embodiments, the solid tumor is at least one of brain tumor, prostate cancer, lung cancer, pancreatic cancer, breast cancer, esophageal cancer, gastric cancer, head & neck cancer and soft tissue sarcomas. According to certain embodiments the solid tumor is a brain tumor selected from a glioblastoma or glioblastoma multiforme and a high-grade intrinsic brain tumor. According to specific embodiments, the brain tumor is glioblastoma multiforme. According to some embodiments, the tumor is a chemotherapy resistant tumor. According to some embodiments, the tumor is a taxane resistant tumor.

According to some embodiments, the present invention provides a method for inhibiting tumor metastasis, comprising administering to a subject with a malignant solid tumor a pharmaceutical composition comprising (a) a particulate biodegradable substrate; (b) a biodegradable polymer; (c) at least one phospholipid having hydrocarbon chains of at least 12 carbons and (d) a taxene, thereby inhibiting tumor metastasis. According to some embodiments, the pharmaceutical composition further comprises a sterol. According to various embodiments the taxene is selected from the group consisting of docetaxel, paclitaxel, derivatives of paclitaxel and cabazitaxel. According to specific embodiments the taxene is docetaxel. According to some embodiments, the pharmaceutical composition is administered to the site of malignant tumor excision site immediately after at least part of the malignant tumor has been removed surgically. According to some embodiments, the solid tumor is at least one of brain tumor, colon carcinoma, prostate cancer, lung cancer, pancreatic cancer, breast cancer, esophageal cancer, gastric cancer, head & neck cancer and soft tissue sarcomas. According to certain embodiments the solid tumor is a brain tumor selected from a glioblastoma or glioblastoma multiforme, a high-grade intrinsic brain tumor and metastasis in the brain originating from other tumors. According to specific embodiments, the brain tumor is glioblastoma multiforme. According to some embodiments, the tumor is a taxane resistant tumor.

The method for treating solid tumors according to some embodiments of the invention provides an adjuvant cancer therapy. The pharmaceutical compositions described herein are intended for local administration to a tumor resection cavity during or shortly after tumor resection surgery, to increase survival rates in cancer patients. The pharmaceutical compositions of the present invention provide prolonged and controlled local exposure to a taxene drug in an intra-operative tumor resection setting, allow for the absorption and distribution of the taxane drug into the local environment of the resected tumor site to provide therapeutic levels of taxane over extended time periods, thereby killing tumor cells left unresected at or near the tumor resection setting, reducing local tumor recurrence and tumor metastatic spreading. The taxane is released from the pharmaceutical compositions beginning immediately after their application to the tumor resection setting and following a zero-order or near zero-order kinetics. The taxane is consistently released for a period of 2-10 weeks, without an initial burst (less than 10% of the taxene embedded within the composition is release within the first 24 hours, typically, less than 8%, 7%, 6%, 5% (w/w) of the taxene is released within the first 24 hours), thus avoiding a potential for toxicity originating from dose dumping (burst effect).

The taxene drug is locally released for a time period ranging from 2-10 weeks; 2-8 weeks; alternatively, 2-6 weeks, alternatively, 2-5 weeks; alternatively, between 2-4 weeks, which is typically the time-lag between tumor resection surgery and initiation of adjuvant radiation therapy, chemotherapy treatment and/or a biological treatment, all of which are typically initiated only after the surgical wound begins the healing process. The disadvantage of the delay in giving adjuvant treatments post tumor removal surgeries, is that cancer cells continue to grow and spread during this time period. The methods and pharmaceutical compositions of the present invention overcome this limitation.

According to some embodiments, the present invention provides neoadjuvant methods for the treatment of solid tumors, comprising intratumoral injection of a pharmaceutical composition comprising (a) a particulate biodegradable substrate; (b) a biodegradable polymer; (c) at least one phospholipid having hydrocarbon chains of at least 12 carbons and (d) a taxene. According to some embodiments, the pharmaceutical composition further comprises a sterol. According to various embodiments the taxene is selected from the group consisting of docetaxel, paclitaxel, derivatives of paclitaxel and cabazitaxel. According to specific embodiments the taxene is docetaxel. According to some embodiments, the solid tumor is at least one of brain tumor, prostate cancer, lung cancer, pancreatic cancer, breast cancer, esophageal cancer, gastric cancer, head & neck cancer and soft tissue sarcomas. The purpose of the neoadjuvant treatment is to reduce the tumor dimensions prior to a surgical procedure for the extraction of the tumor or radiotherapy, thus simplifying the surgical procedure and reducing the risk of cancer cells spreading during the surgical procedure. According to some embodiments, the pharmaceutical composition may be injected directly into the tumor as a dry powder using apparatus suitable for the injection of dry powders. Alternatively, the pharmaceutical composition may be injected as a liquid suspension. According to some embodiments, the tumor is a chemotherapy resistant tumor. According to some embodiments, the tumor is a taxane resistant tumor.

According to some embodiments, the particulate biodegradable substrate used in the pharmaceutical compositions and methods of the invention is composed of particles which are typically spherical or spheroidal. In some embodiments, the particles, which need not be spherical and/or steroidal but preferably are spherical and/or spheroidal, may have an average diameter (as measured by laser diffraction) of at least about 30 μm, at least about 40 μm, at least about 50 μm, at least about 60 μm, at least about 70 μm, at least about 80 μm, at least about 90 μm, at least about 100 μm, between 30 μm and 120 μm, between 30 μm and 100 μm, between 50 μm and 100 μm, not more than about 200 μm, not more than about 180 μm, not more than about 150 μm, not more than about 140 μm, not more than about 130 μm, not more than about 120 μm, not more than about 110 μm, not more than about 100 μm. Each possibility represents a separate embodiment of the invention. According to some embodiments, the particulate substrate used in compositions and methods described herein is a biocompatible, bioabsorbable hydrophilic material, which has low solubility in water such that it is fully eliminated or dissolved in the body within a time period not shorter than 4 weeks, 5 weeks, 6 weeks, 7 weeks, 8 weeks, 9 weeks and preferably not shorter than 10 weeks, and further has a solid shape at ambient temperature and formability. Any materials having these properties may be used without limitation. According to certain embodiments the particulate substrate is composed of tri-calcium phosphate (TCP), preferably 3-TCP. According to other embodiments, the particulate substrate consists of polyvinyl alcohol (PVA), preferably PVA having hydrolysis degree of at least 88%. According to some embodiments, the particulate biodegradable substrate is not calcium sulfate or related hydrates such as calcium dihydrate or calcium sulphate hemihydrate. Without being limited by theory or mechanism of action it is suggested that the polymer-lipid matrix which coats the surface of the biodegradable substrate particles protects the substrate particles from degradation by dissolution. The gradual dissolution of the substrate particles begins only when their surface becomes exposed to body fluids after the degradation of the polymer-lipid matrix. The size of the particles is big enough to ensure that they will not be shifted from the site of administration, at least until most and preferably all the drug has been released. The dimensions of the biodegradable substrate are necessary for ensuring that the pharmaceutical compositions disclosed herein will not migrate from their application site. This is of particular importance when toxic drugs, such as chemotherapy agents are released. Thus, it is important that the overall shape of the particles will not change significantly during the release period of the drug. According to some embodiments, the pharmaceutical compositions used lose between about 10 to 15% of their total weight during the taxane drug release period. The taxane-containing sustained release compositions are designed to anchor into the tissue, preventing their accidental migration over time to other compartments and organs. According to some embodiments, the particulate biodegradable substrate constitutes between about 80-93% (w/w) of the total weight of the pharmaceutical composition.

The biodegradable polymer in pharmaceutical compositions in accordance with embodiments of the invention is a polyester. According to some embodiments, the polyester is selected from the group consisting of polylactic acid (PLA), polyglycolic acid (PGA), polylactic acid-co-glycolic acid (PLGA) and polycaprolactone and any combination or copolymers thereof. According to specific embodiments, the polyester is PLGA. According to some embodiments, the polyester component constitutes 0.5-5% (w/w) of the total weight of the pharmaceutical composition.

According to some embodiments, the phospholipid contains fatty acid chains of at least 12 carbon atoms each. In some embodiments, the fatty acid chains of the phospholipid contain not more than 18 carbon atoms each. In some embodiments, the fatty acid chains of the phospholipid are fully saturated. In some embodiments, at least one of the phospholipid fatty acid chains is non-saturated (e.g. contains at least one double bond). In some embodiments, both phospholipid fatty acid chains are non-saturated. According to some embodiments, the phospholipid having hydrocarbon chains of at least 12 carbons has a phase transition temperature of less than 60° C., less than 55° C., less than 50° C., less than 45° C., less than 42° C., less than 40° C., less than 38° C., less than 35° C., less than 32° C., less than 30° C., less than 28° C., less than 25° C. In some embodiments the phospholipid comprises a phospholipid selected from the group consisting of a phosphatidylcholine, a mixture of phosphatidylcholines, a phosphatidylethanolamine, and combinations thereof. According to some embodiments the second lipid comprises a phosphatidylcholine or a mixture of phosphatidylcholines. In some embodiments, the phosphatidylcholine is selected from the group consisting of DMPC, DPPC, DSPC, DOPC and any combination thereof. In some embodiments, the phosphatidylcholine is selected from DMPC, DPPC, DSPC and any combination thereof. In some embodiments, the phosphatidylcholine is selected from DMPC, DPPC and any combination thereof. In some embodiments, the phosphatidylcholine is selected from DMPC, DSPC and any combination thereof. According to certain embodiments, the phosphatidylcholine is DMPC. In some embodiments, the phospholipid component constitutes 2-15% (w/w) of the total weight of the pharmaceutical composition.

According to some embodiments, the pharmaceutical compositions further comprise a sterol. In some embodiments, the sterol is a phytosterol. In some embodiments, the sterol is a zoosterol. According to specific embodiments, the sterol is a cholesterol. In some embodiments, the sterol constitutes 0-4% (w/w) of the total weight of the pharmaceutical composition. In some preferred embodiments, the sterol is cholesterol and constitutes up to 50% (w/w) of the total lipid content of said pharmaceutical composition. Total lipid content refers to total mass of all the lipids in the pharmaceutical composition (e.g. sterol, phospholipid and any additional lipid additive comprised in the pharmaceutical composition. According to some embodiments the sterol and polymer are non-covalently associated.

According to some embodiments, the taxane is incorporated into the polymer-lipid-based matrix. According to some embodiments the taxene constitutes between 0.2% and 2.6% (w/w) of the total weight of the pharmaceutical composition used in the methods described herein. Alternatively, the taxane constitutes between 0.5% and 1.5% (w/w) of the total weight of the pharmaceutical composition. According to certain embodiments, the taxane constitutes between 0.7% and 1.3% (w/w), alternatively between 0.7% and 1.0% (w/w) of the total weight of the pharmaceutical composition. According to various embodiments the taxane is selected from the group consisting of docetaxel, paclitaxel, derivatives of paclitaxel and cabazitaxel. According to specific embodiments the taxane is docetaxel. According to some embodiments of the methods of the present invention, the pharmaceutical composition is administered to the surface of a solid tumor or to the surface of the resection cavity of a solid tumor following surgical removal of the tumor. According to some embodiments of the methods of the present invention, the pharmaceutical composition is applied to the surface of the solid tumor or the inner surface of the resection cavity at an amount ranging from 20 mg to 260 mg per surface area of 1 cm². According to alternative embodiments, the composition is applied at an amount ranging from 50 mg to 160 mg; 50 mg to 160 mg; between 50 mg to 150 mg; between 50 mg to 120 mg; between 50 mg to 100 mg; 50 mg to 100 mg; between 75 mg to 160 mg; between 75 mg to 120 mg; between 75 mg to 100 mg per 1 cm².

According to some embodiments the pharmaceutical composition is in the form of a powder. According to some embodiments, the powder is spread or sprinkled over the surface of the tumor or applied to the inner surface of the resection cavity. The powder may be additionally or alternatively intratumorally injected using suitable powder injectors. According to certain embodiments of the invention, the pharmaceutical composition is formulated as a paste prior to its application to the tumor site or tumor inner surface of the resection cavity. According to some embodiments, the paste is spread over the surface of the tumor or applied to the inner surface of the resection cavity for example with a spatula. According to additional embodiments, the pharmaceutical composition may be formulated as a suspension for injection.

The method for treating a solid tumor according to some embodiments of the invention comprises administering to a subject with a solid tumor a pharmaceutical composition comprising: (a) tri-calcium phosphate particles; (b) a polyester; (c) a phosphatidylcholine having hydrocarbon chains of at least 12 carbons and (d) a taxane, wherein the composition is intended for local administration to the surface of a solid tumor or to the inner surface of the resection cavity of a solid tumor. According to some embodiments, the composition further comprises cholesterol. According to some embodiments, the taxane is selected from the group consisting of docetaxel, paclitaxel, derivatives of paclitaxel and cabazitaxel. According to specific embodiments the taxane is docetaxel. According to some embodiments, the polyester is PLGA (poly (lactic-co-glycolic acid). According to some embodiments, the phosphatidylcholine hydrocarbon chains are saturated. According to some embodiments, the phosphatidylcholine is 1,2-dimyristoyl-sn-glycero-3-phosphocholine (DMPC). According to some embodiments, the docetaxel constitutes between 0.2% and 2.6% (w/w) of the total weight of the pharmaceutical composition. Alternatively, the docetaxel constitutes between 0.5% and 1.5% (w/w) of the total weight of the pharmaceutical composition. According to certain embodiments, the docetaxel constitutes between 0.7% and 1.3% (w/w), alternatively between 0.7% and 1.0% (w/w) of the total weight of the pharmaceutical composition. According to some embodiments the tri-calcium phosphate (TCP) is selected from the group consisting of α-tri-calcium phosphate, β-tri-calcium phosphate and a combination thereof. According to specific embodiments, the TCP is β-tri-calcium phosphate. According to some embodiments, the pharmaceutical composition is applied to the surface of the solid tumor or the surface of the resection cavity at an amount ranging from 20 mg to 500 mg per surface area of 1 cm². According to alternative embodiments, the composition is applied at an amount ranging from 50 mg to 400 mg, 50 mg to 350 mg, 50 mg to 300 mg, 50 mg to 275 mg, 50 mg to 250 mg, 50 mg to 225 mg, 50 mg to 200 mg, 50 mg to 180 mg, 50 mg to 170 mg; 50 mg to 160 mg; between 50 mg to 150 mg; between 50 mg to 120 mg; between 50 mg to 100 mg; 50 mg to 100 mg; between 75 mg to 160 mg; between 75 mg to 120 mg; between 75 mg to 100 mg per 1 cm². According to some embodiments, the solid tumor is a brain tumor. According to some embodiments, the brain tumor is glioblastoma multiforme. According to some embodiments, the tumor is a taxane resistant tumor.

According to certain embodiments, the present invention provides methods for the treatment of a solid tumor comprising topical administration to the surface of a solid tumor or to the surface of a resection cavity of a solid tumor, a pharmaceutical composition comprising (a) 80-93% (w/w) of tri-calcium phosphate particles; (b) 1%-4.0% (w/w) polyester; (c) 0.0-2.0% (w/w) cholesterol; (d) 4.0-15.0% (w/w) of a phosphatidylcholine having hydrocarbon chains of at least 12 carbons; (e) 0.2-2.6% (w/w) of docetaxel. According to some embodiments, the docetaxel constitutes between 0.5% and 1.5% (w/w) of the total weight of the pharmaceutical composition. According to certain embodiments, the docetaxel constitutes between 0.7% and 1.3% (w/w), alternatively between 0.7% and 1.0% (w/w) of the total weight of the pharmaceutical composition. According to some embodiments, the polyester is PLGA (poly (lactic-co-glycolic acid). According to some embodiments, the phosphatidylcholine hydrocarbon chains are saturated. According to some embodiments, the phosphatidylcholine is 1,2-dimyristoyl-sn-glycero-3-phosphocholine (DMPC). According to some embodiments the tri-calcium phosphate (TCP) is selected from the group consisting of α-tri-calcium phosphate, β-tri-calcium phosphate and a combination thereof. According to specific embodiments, the TCP is β-tri-calcium phosphate. According to some embodiments, the pharmaceutical composition is applied to the surface of the solid tumor or the surface of the resection cavity at an amount ranging from 20 mg to 500 mg per surface area of 1 cm². According to alternative embodiments, the composition is applied at an amount ranging from 50 mg to 400 mg, 50 mg to 350 mg, 50 mg to 300 mg, 50 mg to 275 mg, 50 mg to 250 mg, 50 mg to 225 mg, 50 mg to 200 mg, 50 mg to 180 mg, 50 mg to 170 mg; 50 mg to 160 mg; between 50 mg to 150 mg; between 50 mg to 120 mg; between 50 mg to 100 mg; 50 mg to 100 mg; between 75 mg to 160 mg; between 75 mg to 120 mg; between 75 mg to 100 mg per 1 cm². According to some embodiments, the solid tumor is a brain tumor. According to some embodiments, the brain tumor is glioblastoma multiforme. According to some embodiments, the tumor is a docetaxel resistant tumor.

The pH of the pharmaceutical compositions disclosed herein is inherently provided by the excipients present in the pharmaceutical composition. According to some embodiments, the pH of the pharmaceutical composition is between 7.0 and 9.0 as measured by pH electrode InLab® Solids Go-ISM, preferably between 7.5 and 8.5. According to some embodiments, the pharmaceutical composition further comprises a pH adjustment agent. A pH adjustment agent such as a buffer or an acid can be added to the pharmaceutical composition to maintain the pH to 3.5 to 7; 3.5 to 6.5; 4 to 6; 4 to 5.5; 4 to 5 or 4 to 4.5. Each possibility represents a separate embodiment of the invention. According to some embodiments, maintaining the pH of the pharmaceutical composition below 7, preferably below 6, more preferably between 4 to 5 stabilizes the taxene and slows down the transition of the taxane into its' 7-epimeric impurities during storage. According to certain embodiments, the taxene is docetaxel and the pH of the pharmaceutical composition is between 4 to 5.5. Suitable acids that may be included in the pharmaceutical composition include organic acids such as acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and mixtures thereof as well as inorganic acids such as hydrochloric acid, phosphoric acid, nitric acid, and sulfuric acid, or combinations thereof. Acetic acid is a preferred pH adjustment agent. The amount of the pH adjusting agent in the pharmaceutical composition according to some embodiments is between 0.1-5% (w/w); 0.1-4% (w/w); 0.1-3% (w/w); 0.1-2% (w/w); 0.2-2% (w/w); 0.3-2% (w/w); 0.5-2% (w/w); 0.5-1.8% (w/w); 0.5-1.7% (w/w); 0.5-1.6% (w/w); 0.5-1.5% (w/w); 0.5-1.4% (w/w); 0.5-1.3% (w/w); 0.5-1.2% (w/w); 0.5-1.1% (w/w) or 0.5-1.0% (w/w) of the total weight of the pharmaceutical composition. Each possibility represents a separate embodiment of the invention.

Tissue penetration of chemotherapeutic drugs from the surface of a resected tumor deeper into the cancerous tissue is a major challenge. Although active or passive targeted therapies based on targeted agents or enhanced permeability and retention (EPR) can improve the therapeutic effect of chemotherapy, there are still challenges from the penetrability of nanomedicine in tumor interstitium (Xiaoqian et al. Biomacromolecules 2019, 20:2637-48). To date, in most therapies the active agent(s) have failed to penetrate efficiently into tumor tissues. This challenge is even greater when treating brain tumors. Glioblastoma multiforme is a diffused brain tumor characterized by high infiltration into the brain parenchyma. This process is boosted by the interaction with local (microglia) and infiltrating immune cells (macrophages and Treg cells), which produce cytokines and matrix-degrading enzymes important for tumor growth and expansion into the brain. As a result, it is difficult and almost impossible to completely remove (resect) a GBM tumor by neurosurgery without significantly risking the patient with neurological damage. Thus, despite continuous progress in neurosurgery, GBM infiltrative behaviour interferes with complete tumor resection and is certainly the main reason of the poor clinical outcome for patients. The present invention provides three major factors that improve penetration of the drug from the resected surface into the tissue; (1) high local concentration in immediate proximity to the surface of the tumor resection cavity. (2) prolonged exposure to said high concentration and (3) physical protection of the released chemotherapeutic agent. The high local concentration over an extended period allows the development of a higher driving concentration of the released drug, thereby not only extending the exposure to the drug but further supporting its penetration deeper into the tissue, thereby enabling the eradication of tumor cells that have infiltrated farther from the surface. According to some embodiments, the taxane penetration using the methods and composition disclosed herein extends to a distance of at least 0.5 cm away from the surface of the resected tumor (e.g. the outer boundary of the remaining tumor margin) as measured by quantitative autoradiography. According to some embodiments, the drug penetration extends to a distance of at least 0.6 cm, 0.7 cm, 0.8 cm, 0.9 cm, 1.0 cm, 1.2 cm, 1.3 cm, 1.4 cm, 1.5 cm, 1.6 cm, 1.7 cm, 1.8 cm, 1.9 cm, 2.0 cm, 2.1 cm, 2.2 cm, 2.3 cm, 2.4 cm, 2.5 cm, 2.6 cm, 2.7 cm, 2.8 cm, 2.9 cm, 3.0 cm away from the surface of the resected tumor. According to some embodiments, the drug penetration extends to not less than 2.5 cm away from the surface of the resected tumor, alternatively, not less than 2.4 cm, 2.3 cm, 2.2 cm, 2.1 cm, 2.0 cm, 1.9 cm, 1.8 cm, 1.7 cm, 1.6 cm, 1.5 cm away from the surface of the resected tumor.

Taxanes are relatively large and highly hydrophobic, properties that limit their tissue penetration, with only little drug reaching farther than 100 μm into the tissue (Alastair H. Clin Cancer Res 2007; 13(9): 2804-10). This is at least partially due to the fact that free taxanes become extensively (>98%) bound to circulating proteins and this limit their ability to penetrate into the tissue. The pharmaceutical compositions disclosed herein protect the taxane, not only within the matrix during storage, but also upon release. The taxane is released from the disclosed pharmaceutical composition as upon gradual degradation of the polymer-lipid matrix when maintained in aqueous environments. It has been found that at least 50%, at least 60%, at least 70%, at least 75%, at least 80%, at least 85%, at least 90% of the taxane drug release from the compositions disclosed herein is in association with lipid based colloidal structures, which are formed at the edge of the outer layers of the lipid-polymer based matrix upon exposure to aqueous environment (e.g. body fluids). These lipid based colloidal particles protect the drug from binding to circulating proteins, yet do not harm the drugs' uptake by the tumor cells. Without being limited by theory or mechanism of action, it is suggested that these lipid based colloidal particles improve taxane penetration and infiltration into the tissue.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 shows the accumulated release profiles of docetaxel from pharmaceutical compositions comprising different phospholipids with or without cholesterol, according to several embodiments of the invention.

FIG. 2 shows the amount of docetaxel 7-epimer in docetaxel sustained release compositions comprising different phospholipids with or without cholesterol, according to several embodiments of the invention.

FIG. 3 shows the amount of docetaxel 7-epimer in docetaxel sustained release compositions comprising different amounts of DMPC, according to several embodiments of the invention.

FIGS. 4A and 4B show the effect of the addition of Tween-80 to the docetaxel sustained release compositions comprising DMPC (4A) and DPPC (4B) according to certain embodiments of the invention, on the accumulated release profiles of docetaxel.

FIG. 5 shows the amount of docetaxel 7-epimer in docetaxel sustained release composition comprising various amounts of cholesterol, according to certain embodiments of the invention.

FIG. 6 shows the accumulated release profiles of paclitaxel from paclitaxel sustained release compositions comprising different phospholipids, according to certain embodiments of the invention.

FIG. 7 shows the accumulated release of docetaxel from docetaxel sustained release compositions comprising either PLGA or PEG as the polymer component.

FIG. 8 shows the average tumor volume of CT26 colon carcinoma in BALB/c mice treated locally with various docetaxel sustained release composition according to certain embodiments of the invention.

FIG. 9 shows the average tumor volume of CT26 colon carcinoma in BALB/c mice treated locally with docetaxel sustained release compositions according to certain embodiments of the invention as compared to docetaxel systemic treatment.

FIG. 10 shows a dose response to local treatment with docetaxel sustained release composition comprising 0.87% (w/w) of docetaxel as reflected in the average tumor volume of U87 Glioblastoma multiforme (GBM) tumor in nude mice. Repeated systemic treatment with Gemcitabine served as a positive control.

### DETAILED DESCRIPTION OF EMBODIMENTS OF THE INVENTION

A noted above, the present invention provides methods and sustained release anti-neoplastic compositions for the local treatment of cancer, prevention of cancer recurrence and inhibition of tumor metastasis.

In one aspect of the present invention provides methods for treating a solid tumor, comprising administering to a subject with a solid tumor an effective amount of a pharmaceutical composition comprising a particulate biodegradable substrate coated with a polymer-lipid-based matrix comprising a taxene, wherein the pharmaceutical composition is administered directly to the tumor wall of a resected tumor cavity after tumor has been removed surgically. Alternatively, the pharmaceutical composition may be injected directly into the tumor (e.g. a non-resected tumor, or the tumor leftovers after resection). The methods of the invention are further useful for reducing tumor cell regrowth at a site of solid tumor excision post tumor excision surgery. According to particular embodiments, the methods of the invention are useful for the treatment of a brain tumor (e.g. glioblastoma multiforme). According to some embodiments, the taxene sustained release compositions are intended, according to the methods of the invention for a single application, during tumor excision surgery or at any time before closing the surgical wound.

As used herein, a “solid tumor” (alternatively referred to as “solid cancer”) is an abnormal mass of tissue that usually does not contain cysts or liquid areas. Solid tumors can be either malignant or benign. Malignant solid tumors can invade surrounding tissue and metastasize to new body sides. The term “solid tumor” does not include leukemia (a cancer affecting the blood). Three major types of solid tumors are sarcomas, carcinomas and lymphomas. “Sarcomas” are cancers arising from connective or supporting tissues such as bone or muscle. “Carcinomas” are cancers arising from glandular cells and epithelial cells, which line body tissues. “Lymphomas” are cancers of the lymphoid organs such as the lymph nodes, spleen, and thymus. Exemplary solid tumors include but are not limited to sarcomas and carcinomas such as glioblastoma multiforme, head & neck cancer, colon carcinoma, pancreatic cancer, breast cancer, ovarian cancer, prostate cancer, lung carcinoma, small cell lung carcinoma, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, pancreatic cancer, esophageal cancer, gastric cancer, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatocellular carcinoma, bile duct carcinoma, choriocarcinoma, seminoma, embryonal carcinoma, Wilm's tumor, cervical cancer, testicular tumor, bladder carcinoma, epithelial carcinoma, astrocytoma, medulloblastoma, craniopharyngioma, ependymoma, pinealoma, hemangioblastoma, acoustic neuroma, oligodendroglioma, cutaneous T cell lymphoma (CTCL), melanoma, neuroblastoma, and retinoblastoma.

According to some embodiments, the methods of the invention are useful for the treatment of a brain tumor and for reducing brain tumor cell regrowth at a site of tumor excision post brain tumor excision surgery. Representative examples of brain tumors which may be treated utilizing the compositions and methods described herein include Glial Tumors (such as Anaplastic Astrocytoma, Glioblastoma Multiform, Pilocytic Astrocytoma, Oligodendroglioma, Ependymoma, Myxopapillary Ependymoma, Subependymoma, Choroid Plexus Papilloma); Neuron Tumors (e.g., Neuroblastoma, Ganglioneuroblastoma, Ganglioneuroma, and Medulloblastoma); Pineal Gland Tumors (e.g., Pineoblastoma and Pineocytoma); Menigeal Tumors (e.g., Meningioma, Meningeal Hemangiopericytoma, Meningeal Sarcoma); Tumors of Nerve Sheath Cells (e.g., Schwannoma (Neurolemmoma) and Neurofibroma); Lymphomas (e.g., Hodgkin's and Non-Hodgkin's Lymphoma (including numerous subtypes, both primary and secondary); Malformative Tumors (e.g., Craniopharyngioma, Epidermoid Cysts, Dermoid Cysts and Colloid Cysts); and Metastatic brain tumors (which can be derived from virtually any tumor, the most common being from lung, breast, melanoma, kidney, and gastrointestinal tract tumors).

The term “treatment” or “treating” as used herein refer to an approach for obtaining beneficial or desired results including but not limited to therapeutic benefit and/or a prophylactic benefit. By therapeutic benefit is meant at least one of the following: (a) reducing tumor size; (b) suppressing or reducing tumor growth; (c) reducing or limiting development and/or spreading of metastases; (d) increasing survival or progression-free survival and (e) delaying the time from tumor removal surgery to tumor recurrence.

According to some embodiments, treating the solid tumor comprises inhibiting tumor metastasis. “inhibiting” tumor cell metastasis may comprise any amount of inhibition compared to no treatment.

The term “tumor resection” or “tumor excision” relates to a surgical procedure which goal is to remove the entire tumor or as much of the tumor as possible. While some tumors can be resected easily, others may be located in hard-to-reach locations. Typically, the surgeon removes the tumor with a surrounding amount of normal, healthy tissue (i.e. “surgical margin”) to increase the success of surgery. It will be appreciated by the ones skilled in the art that the removal or resection of the entire tumor by surgery cannot always be achieved. As used herein, the term “tumor resection” refers to a condition in which at least 50%, at least 60%, at least 70%, at least 80%, at least 90% of the tumor volume has been removed by surgery.

The term “tumor resection cavity” as used herein refers to the postoperative defect after tumor resection surgery. Since the entire removal of the tumor is not always achievable by surgery, it is understood that the tumor resection cavity may contain tumor residual mass.

As used herein, the term “effective amount” or “therapeutically effective amount” refers to the amount of a pharmaceutical composition described herein that is sufficient to affect the intended application including but not limited to cancer treatment as defined above. According to some embodiments, the “effective amount” will not exceed the maximum tolerated dose of the taxene used which is defined as the highest dose of a free drug when administered systemically that does not cause unacceptable side effects. According to some preferred embodiments, the “effective amount” in methods of the present invention is lower than the maximum tolerated dose of the taxene. As will be appreciated by the one skilled in the art, the maximum tolerated dose is based on the drug's tolerated systemic toxicity. However, since the systemic exposure to a drug administered locally is significantly lower as compared to the exposure when the drug is administered systemically, the tolerated dose, as defined for local delivery, may be significantly higher as compared to the maximum tolerated dose in a systemic treatment. This is particularly relevant when the drug id release locally without a burst effect.

According to some embodiments of the invention, when the taxane in the pharmaceutical composition is docetaxel, the overall amount of docetaxel administered to a 60 Kg adult in the treatment according to the methods of the invention will not exceed 600 mg, alternatively, will not exceed 500 mg, 450 mg, 400 mg, 350 mg, 300 mg, 290 mg, 280 mg, 270 mg, 260 mg, 250 mg, 240 mg, 230 mg, 220 mg, 210 mg, 200 mg, 190 mg, 180 mg, 170 mg, 160 mg, 155 mg, 150 mg, 145 mg, 140 mg, 135 mg, 130 mg, 125 mg, 120 mg, 115 mg, 110 mg, 100 mg. Each possibility represents a separate embodiment of the invention. According to specific embodiments, the overall dose of docetaxel administered in the treatment according to the methods of the invention will be between 20-600 mg, alternatively between 20-550 mg; 20-500 mg, 20-450 mg, 20-400 mg, 20-350 mg, 20-300 mg, 20-280 mg, 20-260 mg, 20-240 mg, 20-220 mg, 20-200 mg, 20-190 mg, 20-180 mg, 20-170 mg, 20-160 mg, 20-150 mg, 20-140 mg, 20-130 mg, 20-120 mg, 20-110 mg, 20-100 mg, 50-600 mg, 50-550 mg; 50-500 mg, 50-450 mg, 50-400 mg, 50-350 mg, 50-300 mg, 50-280 mg, 50-260 mg, 50-240 mg, 50-220 mg, 50-200 mg, 50-190 mg, 50-180 mg, 50-175 mg, 50-170 mg, 50-165 mg, 50-160 mg, 60-160 mg, 65-160 mg, 70-160 mg, 75-160 mg, 80-160 mg, 85-160 mg, 90-160 mg. 95-160 mg, 100-160 mg, 80-150 mg, 80-140 mg, 80-130 mg, 80-120 mg. Each possibility represents a separate embodiment of the invention.

According to some embodiments of the invention, when the taxane in the pharmaceutical composition is paclitaxel, the overall amount of paclitaxel administered to a 60 Kg adult in the treatment according to the methods of the invention will not exceed 800 mg, alternatively, will not exceed 750, mg, 700 mg, 650 mg, 600 mg, 550 mg, 500 g, 450 mg, 420 mg, 400 mg, 380 mg, 360 mg, 340 mg, 320 mg, 300 mg, 280 mg, 260 mg, 250 mg, 240 mg, 230 mg, 220 mg, 210 mg, 200 mg, 190 mg, 180 mg, 175 mg, 170 mg, 165 mg, 160 mg, 155 mg, 150 mg, 145 mg, 140 mg, 135 mg, 130 mg, 125 mg, 120 mg, 115 mg, 110 mg, 100 mg. Each possibility represents a separate embodiment of the invention. According to specific embodiments, the overall dose of paclitaxel administered in the treatment according to the methods of the invention will be between 60-800 mg, alternatively between 60-750 mg, 60-700 mg, 60-650 mg, 60-600 mg, 60-550 mg, 60-500 mg, 60-450 mg, 60-400 mg, 60-350 mg, 60-320 mg, 60-300 mg, 60-295 mg, 60-290 mg, 60-285 mg, 60-280 mg, 60-275 mg, 60-270 mg, 60-265 mg, 60-260 mg, 60-250 mg, 60-240 mg, 60-230 mg, 60-220 mg, 60-210 mg, 60-200 mg, 60-190 mg, 60-185 mg, 60-180 mg, 60-175 mg, 60-170 mg, 60-165 mg, 60-160 mg, 60-155 mg, 60-150 mg, 80-300 mg, 90-300 mg, 100-300 mg, 110-300 mg, 120-300 mg, 130-300 mg, 140-300 mg, 150-300 mg, 160-300 mg, 170-300 mg, 180-300 mg, 190-300 mg. 200-300 mg, 200-290 mg, 200-280 mg. Each possibility represents a separate embodiment of the invention.

According to some embodiments of the invention, when the taxane in the pharmaceutical composition is cabazitaxel, the overall amount of cabazitaxel administered in the treatment according to the methods of the invention will not exceed 60 mg, alternatively, will not exceed 80 mg, 75 mg, 70 mg, 65 mg, 60 mg, 55 mg, 50 mg, 45 mg, 42 mg, 40 mg, 38 mg, 37 mg, 36 mg, 35 mg, 34 mg, 33 mg, 32 mg, 31 mg, 30 mg, 29 mg, 28 mg, 27 mg, 26 mg, 25 mg, 24 mg, 23 mg, 22 mg, 21 mg, 20 mg. Each possibility represents a separate embodiment of the invention. According to specific embodiments, the overall dose of cabazitaxel administered in the treatment according to the methods of the invention will be between 10-80 mg, alternatively between 10-75 mg, 10-70 mg, 10-65 mg, 10-60 mg, 10-55 mg, 10-50 mg, 10-45 mg, 10-42 mg, 10-40 mg, 10-38 mg, 10-35 mg, 20-50 mg, 20-45 mg, 20-42 mg, 20-40 mg, 20-38 mg, 20-35 mg, 25-50 mg, 25-45 mg, 25-40 mg, 30-50 mg, 30-45 mg, 30-40 mg. Each possibility represents a separate embodiment of the invention. The term “controlled release” refers to control of the rate and/or quantity of taxane drug delivered by the pharmaceutical compositions of the invention. The term “sustained release” means that pharmaceutical active agent is released over an extended period of time.

The pharmaceutical composition disclosed herein are composed of a particulate biodegradable substrate coated or impregnated by a matrix composition comprising (a) a biodegradable polymer, (b) a lipid component comprising at least one phospholipid having fatty acid moieties of at least 12 carbons; and (c) a taxane chemotherapeutic agent. According to some embodiments, the matrix may further comprise a sterol. The matrix compositions provide sustained release of the pharmaceutically active agent at the tumor site or tumor excision site in the body of a subject in need thereof.

In specific embodiments, the polymer and the lipid or lipids form a structurally ordered lipid saturated matrix composition that is substantially free of water. In some embodiments, the matrix composition has a highly organized multilayer structure in which the polymer and lipids are organized in the form of multiple alternating layers. In some embodiments, the matrix comprises at least about 50% total lipids by weight.

According to some embodiments the pharmaceutical composition of the invention comprises between about 80-93% (w/w) of the particulate biodegradable substrate and 7-20% of matrix composition (w/w) of the total weight of the pharmaceutical composition. According to alternative embodiments, the particulate biodegradable substrate constitutes between about 80-92% (w/w), 80-91% (w/w), 80-90% (w/w), 80-89% (w/w), 80-88% (w/w), 80-87% (w/w), 80-86% (w/w), 80-85% (w/w), 81-93% (w/w), 82-93% (w/w), 83-93% (w/w), 84-93% (w/w), 85-93% (w/w), 85-92% (w/w), 85-91% (w/w), 85-90% (w/w), 85-89% (w/w), 85-88% (w/w), 86-89% (w/w) of the total weight of the pharmaceutical composition.

In some embodiments, the matrix composition comprises at least 10% biodegradable polymer by weight of the matrix composition. In some embodiments, the matrix composition comprises between about 10-30% polymer by weight of the matrix composition. In some embodiments, the matrix composition comprises between about 15-25% polymer by weight of the matrix composition. In some embodiments the matrix composition comprises about 20% polymer by weight of the matrix composition. In some embodiments the biocompatible polymer constitutes at least 10% (w/w), at least 11% (w/w), at least 12% (w/w), at least 13% (w/w), at least 14% (w/w), at least 15% (w/w), at least 16% (w/w), at least 17% (w/w), at least 18% (w/w), at least 19% (w/w), at least 20% (w/w), at least 21% (w/w), at least 22% (w/w), at least 23% (w/w), at least 24% (w/w), at least 25% (w/w), at least 26% (w/w), at least 27% (w/w), at least 28% (w/w), at least 29% (w/w), at least 30% (w/w) of the weight of the matrix composition.

According to certain embodiments of the invention, the polymer is a biodegradable polyester. According to some embodiments the polyester is selected from the group consisting of PLA (polylactic acid). “PLA” refers to poly(L-lactide), poly(D-lactide), and poly(DL-lactide). In another embodiment, the polymer is PGA (polyglycolic acid). In another embodiment, the polymer is PLGA (poly(lactic-co-glycolic acid). The PLA contained in the PLGA may be any PLA known in the art, e.g. either enantiomer or a racemic mixture. The PLGA of methods and compositions of the present invention has, in another embodiment, a 50:50 lactic acid/glycolic acid ratio. In another embodiment, the ratio is 60:40. In another embodiment, the ratio is 75:25. In another embodiment, the ratio is 85:15. In another embodiment, the ratio is 90:10. In another embodiment, the ratio is 95:5. In another embodiment, the ratio is another ratio appropriate for an extended or sustained in vivo release profile. The PLGA may be either a random or block copolymer. Each possibility represents a separate embodiment of the present invention. It is to be emphasized that the polymer may be of any size or length (i.e of any molecular weight).

In another embodiment, the biodegradable polyester may be selected from the group consisting of polycaprolactone, polyhydroxyalkanoate, polypropylenefumarate, polyorthoester, polyanhydride, and polyalkylcyanoacrylate, provided that the polyester contains a hydrogen bond acceptor moiety. In another embodiment, the biodegradable polyester is a block copolymer containing a combination of any two monomers selected from the group consisting of a PLA, PGA, a PLGA, polycaprolactone, a polyhydroxyalkanoate, a polypropylenefumarate, a polyorthoester, a polyanhydride, and a polyalkylcyanoacrylate. In another embodiment, the biodegradable polyester is a random copolymer containing a combination of any two of the monomers listed above. Each possibility represents a separate embodiment of the present invention.

The term “biodegradable” refers to a substance that will degrade over time by hydrolytic action, by the action of enzymes and/or by other similar mechanisms in the human body. “Biodegradable” further includes that a substance can break down or degrade within the body to non-toxic components after or while a therapeutic agent has been or is being released.

According to some embodiments, the matrix composition comprises at least about 30% (w/w of the total weight of the matrix composition) of a lipid component comprising at least one phospholipid having fatty acid moieties of at least 12 carbons. According to some embodiments, the matrix composition comprises at least about 40% (w/w) of a lipid component comprising at least one phospholipid having fatty acid moieties of at least 12 carbons, preferably between 12 and 18 carbons, preferably wherein the hydrocarbon chains are fully saturated. According to some embodiments, the matrix composition comprises about 40-75% (w/w) of a lipid component comprising at least one phospholipid having fatty acid moieties of at least 12 carbons. According to some embodiments, the matrix composition comprises about 50-70% (w/w) of a lipid component comprising at least one phospholipid having fatty acid moieties of at least 12 carbons. According to certain typical embodiments, the matrix composition comprises about 60% (w/w) a lipid component comprising at least one phospholipid having fatty acid moieties of at least 12 carbons. In some embodiments, the lipid component comprising at least one phospholipid having fatty acid moieties of at least 12 carbons constitute at least 40% (w/w), at least 45% (w/w), at least 50% (w/w), at least 55% (w/w), at least 60% (w/w), at least 65% (w/w), or at least 70% (w/w) of the total weigh of the matrix composition. In some embodiments, the lipid component comprising at least one phospholipid having fatty acid moieties of at least 12 carbons constitute not more than 75% (w/w), not more than 70% (w/w), not more than 65% (w/w) of the total weight of the matrix composition. According to some embodiments, the lipid component comprises at least one phospholipid molecule having fatty acid moieties of at least 14 carbons. According to some embodiments, the second lipid component comprises at least one phosphatidylcholine molecules having fatty acid moieties of at least 14 carbons. According to some preferred embodiments, the phosphatidylcholine molecules of the composition comprise DMPC. According to some embodiments, the phosphatidylcholine molecules of the composition comprise DPPC. According to some embodiments, the phosphatidylcholine molecules of the composition comprise DSPC. According to some embodiments, the matrix composition comprises DOPC. According to some embodiments, the matrix composition comprises a mixture of DMPC with a second phospholipid having fatty acid moieties of at least 14 carbons. According to some embodiments, the matrix composition comprises a mixture of DMPC and DPPC. Typically, the ratio between DMPC and DPPC in the matrix formulation is between about 10:1 to 1:10. According to some embodiments, the matrix composition comprises about 50-70% (w/w) of DMPC or a mixture of DMPC and DPPC.

According to some embodiments, the sustained release matrix composition may further comprise a sterol. According to some embodiments, the sterol comprises up to 40% (w/w) of total weigh of the matrix composition. According to some embodiments, when present the sterol is non-covalently associated with the biodegradable polymer. According to some embodiments, the sterol constitutes up to about 30% (w/w) of the total weight of the matrix composition. According to some embodiments, the sterol constitutes about 5-40% (w/w), about 5-30% (w/w), about 5-20% (w/w), about 5-15% (w/w), about 7-13% (w/w), about 9-11% (w/w) of the total weight of the matrix composition. According to certain typical embodiments, the matrix composition comprises about 10% (w/w of the total weight of the matrix composition) of sterol. In some embodiments the sterol constitutes at least 5% (w/w), at least 6% (w/w), at least 7% (w/w), at least 8% (w/w), at least 9% (w/w), at least 10% (w/w), at least 11% (w/w), at least 12% (w/w), at least 13% (w/w), at least 14% (w/w), at least 15% (w/w), at least 16% (w/w), at least 17% (w/w), at least 18% (w/w), or at least 19% (w/w) of the matrix. In some embodiments, sterol constitutes not more than 20% (w/w), not more than 19% (w/w), not more than 18% (w/w), not more than 17% (w/w), not more than 16% (w/w), not more than 15% (w/w), not more than 14% (w/w), not more than 13% (w/w), not more than 12% (w/w), not more than 11% (w/w), not more than 10% (w/w), not more than 9% (w/w), not more than 8% (w/w), not more than 7% (w/w), not more than 6% (w/w), or not more than 5% (w/w) of the matrix composition. Each possibility represents a separate embodiment of the present invention. According to certain preferred embodiments, the sterol is cholesterol.

In some embodiments, the lipid:polymer weight ratio in the pharmaceutical composition of the present invention is between 1:1 and 9:1 inclusive. In another embodiment, the ratio is between 2:1 and 9:1 inclusive. In another embodiment, the ratio is between 3:1 and 9:1 inclusive. In another embodiment, the ratio is between 4:1 and 9:1 inclusive. In another embodiment, the ratio is between 5:1 and 9:1 inclusive. In another embodiment, the ratio is between 6:1 and 9:1 inclusive. In another embodiment, the ratio is between 7:1 and 9:1 inclusive. In another embodiment, the ratio is between 8:1 and 9:1 inclusive. In another embodiment, the ratio is between 1.5:1 and 9:1 inclusive. Each possibility represents a separate embodiment of the present invention.

It is to be emphasized that the sustained release period using the compositions of the present invention can be programmed taking into account the biochemical and/or biophysical properties of the polymer and the lipid. Specifically, the degradation rate of the polymer and the fluidity of the lipid should be considered. For example, a PLGA (85:15) polymer will degrade slower than a PLGA (50:50) polymer. A phosphatidylcholine (12:0) is more fluid (less rigid and less ordered) at body temperature than a phosphatidylcholine (18:0). Thus, for example, the release rate of a drug incorporated in a matrix composition comprising PLGA (85:15) and phosphatidylcholine (18:0) will be slower than that of a drug incorporated in a matrix composed of PLGA (50:50) and phosphatidylcholine (14:0). Another aspect that will determine the release rate is the physical characteristics of the entrapped or impregnated drug. In addition, the release rate of drugs can further be controlled by the addition of other lipids into the matrix formulation, some of which are described below.

In various embodiments, the taxane chemotherapeutic drug embedded in the matrix composition coating the particulate substrate may be any suitable taxane, including but not limited to paclitaxel, docetaxel, cabazitaxel, taxadiene, baccatin II, taxchinin A, brevifoliol, taxuspine D, combinations thereof, or pharmaceutically acceptable salts thereof. According to various embodiments, the taxane is docetaxel. According to various embodiments, the taxane is paclitaxel. According to some embodiments, the taxane constitutes between about 3-20% (w/w) of the total weight of the matrix composition. According to some embodiments, the taxane constitutes between about 3-19% (w/w), 3-18% (w/w), 3-17% (w/w), 3-16% (w/w), 3-15% (w/w), 3-14% (w/w), 3-13% (w/w), 3-12% (w/w), 3-11% (w/w), 3-10% (w/w), 3-9% (w/w), 3-8% (w/w), 4-15% (w/w), 4-14% (w/w), 4-13% (w/w), 4-12% (w/w), 4-11% (w/w), 4-10% (w/w), 4-9% (w/w), 4-8% (w/w), 5-15% (w/w), 5-14% (w/w), 5-13% (w/w), 5-12% (w/w), 5-11% (w/w), 5-10% (w/w), 5-9% (w/w), 5-8% (w/w), 6-15% (w/w), 6-14% (w/w), 6-13% (w/w), 6-12% (w/w), 6-11% (w/w), 6-10% (w/w), 6-9% (w/w), 6-8% (w/w) of the total weight of the matrix composition. According to certain embodiments, taxane constitutes between about 0.2% and 2.6% (w/w) of the total weight of the pharmaceutical composition. Alternatively, between about 0.3-2.5%, 0.3-2.4%, 0.3-2.3%, 0.3-2.2%, 0.3-2.1%, 0.3-2.0%, 0.3-1.9%, 0.3-1.8%, 0.3-1.7%, 0.3-1.6%, 0.3-1.5%, 0.3-1.4%, 0.3-1.3%, 0.3-1.2%, 0.3-1.1%, 0.3-1.0%, 0.3-0.0%, 0.5-2.5%, 0.5-2.4%, 0.5-2.3%, 0.5-2.2%, 0.5-2.1%, 0.5-2.0%, 0.5-1.9%, 0.5-1.8%, 0.5-1.7%, 0.5-1.6%, 0.5-1.5%, 0.5-1.4%, 0.5-1.3%, 0.5-1.2%, 0.5-1.1%, 0.5-1.0%, 0.6-2.5%, 0.6-2.4%, 0.6-2.3%, 0.6-2.2%, 0.6-2.1%, 0.6-2.0%, 0.6-1.9%, 0.6-1.8%, 0.6-1.7%, 0.6-1.6%, 0.6-1.5%, 0.6-1.4%, 0.6-1.3%, 0.6-1.2%, 0.6-1.1%, 0.6-1.0%, 0.6-0.9%, 0.7-2.5%, 0.7-2.4%, 0.7-2.3%, 0.7-2.2%, 0.7-2.1%, 0.7-2.0%, 0.7-1.9%, 0.7-1.8%, 0.7-1.7%, 0.7-1.6%, 0.7-1.5%, 0.7-1.4%, 0.7-1.3%, 0.7-1.2%, 0.7-1.1%, 0.7-1.0%, 0.7-0.9%, 0.8-1.0%, 0.8-0.9% (w/w) of the total weight of the pharmaceutical composition. Each possibility represents a separate embodiment of the invention. According to some embodiments the taxane is paclitaxel. According to some embodiments the taxane is docetaxel.

According to some embodiments, the particulate biodegradable substrate used in the pharmaceutical composition and methods of the invention is composed of particles which are typically spherical or steroidal. In some embodiments, the particles, which need not be spherical and/or steroidal but preferably are spherical and/or spheroidal, may have an average diameter (as measured by laser diffraction for example by laser diffraction using a Mastersizer 3000 instrument by Malvern) of at least about 30 μm, at least about 40 μm, at least about 50 μm, at least about 60 μm, at least about 70 μm, at least about 80 μm, at least about 90 μm, at least about 100 μm, between 30 μm and 120 μm, between 30 μm and 100 μm, between 50 μm and 100 μm, not more than about 150 μm, not more than about 140 μm, not more than about 130 μm, not more than about 120 μm, not more than about 110 μm, not more than about 100 μm. Each possibility represents a separate embodiment of the invention. According to some embodiments, the particulate substrate used in compositions and methods described herein is a bioabsorbable hydrophilic material, which has biocompatibility (that is, is low in toxicity, shows only low foreign body reactions in the living body, and may have a good affinity with the body tissue), bioabsorbability (i.e. biodegradability), and hydrophilicity, but which has low solubility in water such that it is fully eliminated or dissolved in the body within a time period not shorter than 4 weeks, not less than 6 weeks, not less than 8 weeks and preferably, not less than 10 weeks, and further has a solid shape at ambient temperature and formability. Any materials having these properties may be used without limitation. According to some embodiments, the biodegradable substrate is selected from the group consisting of hydroxyapatite, carbonated calcium hydroxyapatite, α-tricalcium phosphate (α-TCP), β-tricalcium phosphate (β-TCP), amorphous calcium phosphate, tetracalcium phosphate, anhydrous dicalcium phosphate, anhydrous monocalcium phosphate, octocalcium phosphate, disodium hydrogen phosphate, and other phosphate salt-based bioceramics and combination thereof. According to some embodiments the particulate substrate is composed of tri-calcium phosphate (TCP), preferably β-TCP. According to other embodiments, the particulate substrate consists of polyvinyl alcohol (PVA), preferably PVA having hydrolysis degree of at least 88%. According to some embodiments, the biodegradable substrate is a porous substrate having a porosity ranging from 40-80%, 45-80%, 50-80%, 55-80%, 60-80%, 65-80%, 65-75%. Each possibility represents a separate embodiment of the invention.

The term “average diameter size” as used herein, means that at least about 50% of the substrate particles have a size of less than the measured average diameter size as measured by laser diffraction. By way of example, a particle having an average particle size of 100 μm means that at least about 50% of the particles have a diameter of less than 100 μm.

In specific embodiments, the pharmaceutical composition is substantially free of water. “Substantially free of water” as used herein refers, in one embodiment, to a pharmaceutical composition containing less than 2% water by weight of the total weight of the pharmaceutical composition. In another embodiment, the term refers to a matrix composition containing less than 1.5% water, less than 1.4% water, less than 1.3% water, less than 1.2% water, less than 1.1% water, less than 1.0% water, less than 0.9% water, less than 0.8%, less than 0.7%, less than 0.6%, less than 0.5% of water by weight of the total weight of the pharmaceutical composition. In another embodiment, the term refers to the absence of amounts of water that affect the water-resistant properties of the matrix composition. In another embodiment, the term refers to a pharmaceutical composition manufactured without the use of any aqueous solvents. In another embodiment, producing the pharmaceutical composition using a process substantially free of water, as described herein, enables lipid saturation. Lipid saturation confers upon the matrix composition ability to resist bulk degradation in vivo; thus, the matrix composition exhibits the ability to mediate extended release on a scale of several days to several weeks (up to about 10 weeks). The total amount of water in the composition may be determined by any method known in the art such as Karl Fischer and loss on drying.

### Technology Platform of the Pharmaceutical Composition Used in Methods of the Present Invention

According to some embodiments, the sustained release matrix composition, coating the particulate biodegradable substrate, has a highly organized multilayer structure in which the polymer forms one type of layer, the phospholipids form a second type of layer, and the two types of layers are organized in the form of multiple alternating or quasi-alternating layers. According to some embodiments, the matrix composition comprises a continuous structure devoid of internal gaps and/or free volume. The coating matrix composition is lipid-saturated, indicating that the space between the polymer layers or polymer backbone is filled with lipid molecules in combination the taxane drug to the extent that additional lipid moieties can no longer be incorporated into the matrix to an appreciable extent.

The coating matrix compositions disclosed herein are lipid saturated. “Lipid saturated,” as used herein, refers to saturation of the polymer of the matrix composition with the lipid component (e.g. phospholipids and optionally a sterol) in combination with the taxane drug present in the matrix, and any other lipids that may be present. The matrix composition is saturated by whatever lipids are present. In another embodiment, “lipid saturation” refers to filling of internal gaps (free volume) within the lipid matrix as defined by the external border of the polymeric backbone. The gaps are filled with phosphatidylcholines optionally in combination with cholesterol and possibly other type of lipids and the taxane drug present in the matrix, to the extent that additional lipid moieties can no longer be incorporated into the matrix to an appreciable extent. Lipid-saturated matrices of the present invention exhibit the additional advantage of not requiring a synthetic emulsifier or surfactant such as polyvinyl alcohol; thus, matrix compositions of the present invention are typically substantially free of polyvinyl alcohol.

In some embodiments, the matrix composition is capable of releasing at least 40% of the taxane drug at zero-order kinetics when it is exposed to an aqueous medium and further maintained in an aqueous medium. In some embodiments, at least 50%, at least 55%, at least 60% of the taxane is released from the matrix composition at zero-order kinetics when it is maintained in an aqueous medium. Without being limited by a specific theory or mechanism of action it is suggested that the organized structure or substructure of the matrix composition of the invention is one of the main reasons for the zero-order release rate of the drug or drugs from the matrix formulation following its hydration. Thus, the zero order release rate may be attributed to slow and continuous “peeling” of the hydrated surface layer(s) of the highly organized layers of lipids and polymer, with concomitant release of the taxane drug as the components of the surface layer are removed from the matrix. It is surmised that this process slowly repeats itself, releasing the taxane drug at a steady rate over days and weeks, until the matrix has been completely degraded. Without wishing to be bound by theory, it is believed that the polymer forms a first type of layer, and that the phospholipid(s) forms a second type of layer, and that these layers alternate i.e. (polymer)-(phospholipid)-(polymer)-(phospholipid); the term “quasi-alternation” as used herein to refer to the situation in which there is alternation of more than one instance of a type of layer, e.g. (polymer)-(phospholipid)-(phospholipid)-(polymer)-(phospholipid)-(phospholipid)-(polymer).

In some embodiments, the matrix composition has multiple mixed layers of polymer and phospholipid as described above and it is not in the form of a microsphere, a micelle, a reversed micelle or a liposome. In some embodiments, the matrix composition does not comprise micelles, reverse micelles or liposomes.

According to some embodiments the matrix of the present invention is water resistant. As such water cannot easily, if at all, diffuse into the inner layers of the matrix and the taxane drug entrapped between the inner layers cannot easily, if at all, diffuse out of the matrix. More particularly it refers to a composition having its bulk (e.g. part of the composition which is surrounded by an external surface, said external surface is exposed to the surrounding environment) not exposed to water, or exposed to the extent that the amount of penetrating water is small and insufficient to cause matrix bulk disintegration or degradation. Without wishing to be bound by theory or mechanism of action, the water resistance properties of the matrix composition, together with its unique multilayered structure confer the matrix with its sustained release properties, e.g. its ability to release at least 40%, preferably at least 50%, 60% or at least 70% of the taxane chemotherapeutic drug from the composition at zero order kinetics for periods of time ranging from several days to several weeks and even months, when the composition is maintained in an aqueous environment at physiological temperature.

The efficacy of a drug is commonly determined by its local concentration. That, in turn, is determined by the ratio between the accumulation rate of drug released from the product vs. its elimination by physical distribution to surrounding tissue, as well as by neutralization and/or degradation. An optimal drug delivery system should release the drug according to the biological need, in order to create an effective concentration at close or immediate proximity to the target and throughout a sufficient period of time needed for the desired biological effect. This can be achieved by releasing the drug at the target site at a rate that will result in an effective concentration that is above the minimal effective concentration, and preferably below the toxic level and for the desired period of time needed for effective therapeutic effect. It has been surprisingly found that the pharmaceutical compositions according to some embodiments of the invention were capable to treat a solid tumor and inhibit its local recurrence after tumor excision surgery even when the overall amount of drug (e.g. docetaxel) administered (embedded in the pharmaceutical composition), was less than 30% of the maximum tolerated dose of the drug base on the prescribing Information of the drug. Yet further, a similar outcome has been obtained even when the tumor was a taxane resistant tumor.

One of the advantages of the compositions and methods of the present invention is their ability to control the local exposure to the taxane drug by controlling the taxane supply rate to the site. The supply rate is dictated by 1) the taxane release profile, 2) the release rate and 3) the duration of release. These parameters are closely related; while the release rate is strongly depended on the specific formulation, the duration is a function of two factors: release rate and the size of drug reservoir. The pharmaceutical compositions of the invention comprising a combination of specific lipids and polymers loaded with a taxane drug, preferably docetaxel, determines not only the release rate profile of the taxane, but also allows control over the release rate during a prolonged zero-order kinetic phase. Without wishing to be bound by theory or mechanism of action it is suggested that the most effective and safe release profile of a chemotherapeutic drug will be a continuous, zero order kinetics, release over sufficient duration, without an initial burst, for example up to 14 days, up to 15 days, up to 16 days, up to 17 days, up to 18 days, up to 19 days, up to 20 days, up to 21 days, up to 22 days, up to 23 days, up to 24 days, up to 25 days, up to 26 days, up to 27 days, up to 28 days, up to 29 days, up to 30 days, up to 31 days, up to 32 days, up to 33 days, up to 34 days, up to 35 days, up to 36 days, up to 37 days, up to 38 days, up to 39 days, up to 40 days, up to 6 weeks, up to 7 weeks, up to 8 weeks, up to 9 weeks, up to 10 weeks, preferably between about 14-35 days.

“Zero-order release rate” or “zero order release kinetics” means a constant, linear, continuous, sustained and controlled release rate of the taxane from the pharmaceutical composition, i.e. the plot of amounts of the taxane released vs. time is linear. According to some embodiments, at least 40% preferably, at least 50% and more preferably, at least 60% of the taxane is released from the composition at zero order kinetics at a rate between about 1-7%, 1-6%, 1-5%, 1-4%, 1-3%, 2-7%, 2-6%, 2-5%, 2-4%, 2-3% (weight percent of the taxane released per day/total weight of the taxane initially encapsulated in the composition), each possibility represent a separate embodiment of the invention.

According to some embodiments, when maintained in an aqueous medium at physiological temperatures, 1 to 10% of said taxane is released from the composition by the end of the first day, 10 to 50% of the taxane is released from the composition by the end of the first week, 20 to 100% of the taxane is released from the composition by the end of the first two weeks and 30 to 100% of the taxane is released by the end of the first three weeks. In some embodiments, when maintained in an aqueous medium at physiological temperatures, at least 10% but not more than 50% of the taxane is released by the end of the first week, at least 20%, but not more than 80% of the taxane is released by the end of the second week, at least 30% of the taxane is released by the end of the third week. At least 40% of the taxane is released by the end of the third week. At least 50% of the taxane is released by the end of the third week. At least 60% of the taxane is released by the end of the third week. According to currently preferred embodiments, the taxane is docetaxel.

The pharmaceutical compositions used in methods of the present invention release the taxane locally at the tumor site or at the tumor excision site at a predictable, long-term release. Thus, taxane drug levels can be maintained locally at the tumor site, while maintaining low or no systemic levels. Due to the prolonged local release of the taxane, a safe dose of local taxane, typically smaller than a single dose commonly administered by I.V., is highly effective in treating the tumor and preventing its recurrence. By way of example, the amount of docetaxel in 10 grams of the pharmaceutical composition used in methods of the present invention (wherein the docetaxel constitutes between about 0.7-1% of the total weight of the composition) suitable for the application to the surface of a tumor resection cavity having a diameter of about 5 cm (estimated cavity surface of about 25 cm²) is about 50% of the amount of docetaxel recommended for a single dose commonly administered I.V once every three weeks.

Additionally, the pharmaceutical composition acts like a reservoir in which the entrapped taxane is protected. In contrast to the conventional polymer-based delivery systems, this characteristic can protect sensitive drugs reservoir not only from biological degradation agents such as enzymes, but also from chemical destruction due to in vivo soluble materials and hydration. When prolong effect is needed, this characteristic is becoming highly important.

### Therapeutic Methods

The methods of the invention directed at treating solid tumors and preventing their recurrence after tumor excision surgeries address medical needs that are currently lacking effective solutions and that are of great concern to the medical community. The methods of the invention provide localized tumor treatment and prevention of tumor recurrence to be applied directly to the tumor excision site cavity during or immediately after tumor excision surgery or as a neoadjuvant therapy by intratumoral injection directly into the tumor. The methods of the invention are suitable for cancer treatment, prevention of cancer recurrence and cancer metastasis in a variety of solid tumors.

According to some embodiments the present invention provides a method for treating a brain tumor, comprising the step of administering to the surface of a solid brain tumor or to the surface of a resection cavity of a solid brain tumor after its' excision, a therapeutically effective amount of a pharmaceutical composition comprising: (a) a particulate biodegradable substrate; (b) a biodegradable polymer; (c) at least one phospholipid having hydrocarbon chains of at least 12 carbons and (d) a taxene. According to some embodiments, the brain tumor is glioblastoma multiforme. According to some embodiments, the pharmaceutical composition further comprises a sterol. According to various embodiments the taxene is selected from the group consisting of docetaxel, paclitaxel, derivatives of paclitaxel and cabazitaxel. According to specific embodiments the taxene is docetaxel. According to some embodiments, the biodegradable polymer is a polyester. According to some embodiments, the biodegradable polymer is PLGA. According to some embodiments, the phospholipid is a phosphatidylcholine having hydrocarbon chains of between 12 and 18 carbons. According to certain embodiments, the phospholipid component comprises DMPC. According to some embodiments, the pharmaceutical composition used in methods for treating a brain tumor comprises (a) 80-93% (w/w) of tri-calcium phosphate; (b) 1%-4.0% (w/w) PLGA; (c) 0.0-2.0% (w/w) cholesterol; (d) 4.0-15.0% (w/w) of DMPC; (e) 0.2-2.6% (w/w) of docetaxel. According to some embodiments, the docetaxel constitutes between 0.5% and 1.5% (w/w) of the total weight of the pharmaceutical composition. According to certain embodiments, the docetaxel constitutes between 0.7% and 1.3% (w/w), alternatively between 0.7% and 1.0% (w/w) of the total weight of the pharmaceutical composition. According to some embodiments the tri-calcium phosphate (TCP) is selected from the group consisting of α-tri-calcium phosphate, β-tri-calcium phosphate and a combination thereof. According to specific embodiments, the TCP is β-tri-calcium phosphate. According to some embodiments, the pH of the pharmaceutical composition is between about 7.5 and 8.5. According to some embodiments, the pharmaceutical composition for the treatment of brain cancer further comprising a pH adjusting agent. According to some embodiments the pH of the pharmaceutical composition is between about 4 to 6. According to some embodiments, a pH of 4 to 6 stabilizes the taxane (e.g. docetaxel) and reduces its transformation to it 7-epimer. According to certain embodiments, the method for the treatment of a brain tumor comprises topical administration of the pharmaceutical compositions disclosed above to the surface of a solid brain tumor or to the surface of a resection cavity of a solid brain tumor after its' excision. According to some embodiments, excision of a brain tumor as used herein refers to a condition wherein at least 50%, at least 60%, at least 70%, at least 80%, at least 90% of the tumor volume has been removed by surgery. In cases where the brain tumor is not accessible by surgery and cannot be resected or when the patient bearing the tumor is non-operable due to its medical condition, the pharmaceutical composition may be injected directly into the tumor. According to certain embodiments, the pharmaceutical comprises (a) 85-92% (w/w) of tri-calcium phosphate; (b) 2.0%-3.0% (w/w) PLGA; (c) 0.0-2.0% (w/w) cholesterol; (d) 4.0-10.0% (w/w) of DMPC and (e) 0.5-1.5% (w/w) of docetaxel. According to some exemplary embodiments the pharmaceutical composition comprises (a) 86-89% (w/w) of tri-calcium phosphate; (b) 2.4%-2.8% (w/w) PLGA; (c) 0.8-1.5% (w/w) cholesterol; (d) 7.0-9.0% (w/w) of DMPC; and (e) 0.6-1.3% (w/w) of docetaxel. According to some embodiments, the tri-calcium phosphate is 3-tri-calcium phosphate. The methods disclosed above for the treatment of brain tumors reduce, minimize or effectively eliminate the delay between the removal of the tumor by surgery and the initiation of currently implemented adjuvant therapies such as radiation and systemic chemotherapy, which are typically given about 4 weeks post-surgery and only after the surgical wound has begun the healing process. According to some embodiments the methods of the present invention for the treatment of brain tumors further inhibit the formation of tumor metastasis.

According to some embodiments the method disclosed above is suitable for the treatment of a primary brain tumor. Primary brain tumor can arise from different type of brain cells or the membranes around the brain (meninges), nerves or glands. The most common type of primary tumors in the brain is glioma, which arises from the glial tissue of the brain. According to some embodiments the glioma is astrocytoma. According to some embodiments astrocytoma is selected from the group consisting of grade I (pilocytic) astrocytoma, grade II (fibrillary) astrocytoma, grade III (anaplastic) astrocytoma and grade IV glioblastoma multiforme (GBM). According to other embodiments, the glioma is oligodendroglioma. According to yet further embodiments the glioma is ependymomas. According to some embodiments, the brain tumor is a secondary or metastatic brain tumor. A secondary or metastatic brain tumor is generated by cancer cells that migrate from tumors developed in other parts of the body. The most common brain metastases originated from lung cancer cells, breast cancer cells, melanoma, colorectal and kidney cancer cells.

According to some embodiments the present invention provides a method for treating a colon carcinoma, comprising the step of administering to the surface of a solid colon carcinoma tumor or to the surface of a resection cavity of a solid carcinoma tumor after its' excision, a therapeutically effective amount of a pharmaceutical composition comprising: (a) a particulate biodegradable substrate; (b) a biodegradable polymer; (c) at least one phospholipid having hydrocarbon chains of at least 12 carbons and (d) a taxene. According to some embodiments, the pharmaceutical composition further comprises a sterol. According to various embodiments the taxene is selected from the group consisting of docetaxel, paclitaxel, derivatives of paclitaxel and cabazitaxel. According to specific embodiments the taxene is docetaxel. According to some embodiments, the biodegradable polymer is a polyester. According to some embodiments, the biodegradable polymer is PLGA. According to some embodiments, the phospholipid is a phosphatidylcholine having hydrocarbon chains of between 12 and 18 carbons. According to certain embodiments, the phospholipid component comprises DMPC. According to some embodiments, the pharmaceutical composition used in methods for treating colon carcinoma comprises (a) 80-93% (w/w) of tri-calcium phosphate; (b) 1%-4.0% (w/w) PLGA; (c) 0.0-2.0% (w/w) cholesterol; (d) 4.0-15.0% (w/w) of DMPC; (e) 0.2-2.6% (w/w) of docetaxel. According to some embodiments, the docetaxel constitutes between 0.5% and 1.5% (w/w) of the total weight of the pharmaceutical composition. According to certain embodiments, the docetaxel constitutes between 0.7% and 1.3% (w/w), alternatively between 0.7% and 1.0% (w/w) of the total weight of the pharmaceutical composition. According to some embodiments the tri-calcium phosphate (TCP) is selected from the group consisting of α-tri-calcium phosphate, β-tri-calcium phosphate and a combination thereof. According to specific embodiments, the TCP is β-tri-calcium phosphate. According to some embodiments, the pH of the pharmaceutical composition is between about 7.5 and 8.5. According to some embodiments, the pharmaceutical composition for the treatment of colon carcinoma further comprises a pH adjusting agent. According to some embodiments the pH of the pharmaceutical composition is between about 4 to 6. According to some embodiments, a pH of 4 to 6 stabilizes the taxane (e.g. docetaxel) and reduces its transformation to it 7-epimer. According to certain embodiments, the method for the treatment of a colon carcinoma tumor comprises topical administration of the pharmaceutical compositions disclosed above to the surface of a solid colon tumor or to the surface of a resection cavity of a colon carcinoma tumor after its' excision. According to some embodiments, excision of a colon carcinoma tumor as used herein refers to a condition wherein at least 50%, at least 60%, at least 70%, at least 80%, at least 90% of the tumor volume has been removed by surgery. In cases where the tumor is not accessible by surgery and cannot be resected or when the patient bearing the tumor is non-operable due to its medical condition, the pharmaceutical composition may be injected directly into the colon tumor. According to certain embodiments, the pharmaceutical comprises (a) 85-92% (w/w) of tri-calcium phosphate; (b) 2.0%-3.0% (w/w) PLGA; (c) 0.0-2.0% (w/w) cholesterol; (d) 4.0-10.0% (w/w) of DMPC and (e) 0.5-1.5% (w/w) of docetaxel. According to some exemplary embodiments the pharmaceutical composition comprises (a) 86-89% (w/w) of tri-calcium phosphate; (b) 2.4%-2.8% (w/w) PLGA; (c) 0.8-1.5% (w/w) cholesterol; (d) 7.0-9.0% (w/w) of DMPC; and (e) 0.6-1.3% (w/w) of docetaxel. According to some embodiments, the tri-calcium phosphate is β-tri-calcium phosphate. According to some embodiments the methods of the present invention for the treatment of colon carcinoma further inhibit the formation of tumor metastasis. According to additional embodiments, the method for the treatment disclosed above for threating colon carcinoma is suitable also for the treatment of prostate cancer, lung cancer, pancreatic cancer, breast cancer, esophageal cancer, gastric cancer, head & neck cancer and soft tissue sarcomas.

According to some embodiments, the present invention provides a method for inhibiting tumor metastasis, comprising administering to a subject with a malignant solid tumor a pharmaceutical composition comprising (a) a particulate biodegradable substrate; (b) a biodegradable polymer; (c) at least one phospholipid having hydrocarbon chains of at least 12 carbons and (d) a taxene, thereby inhibiting tumor metastasis. According to some embodiments, the pharmaceutical composition further comprises a sterol. According to various embodiments the taxene is selected from the group consisting of docetaxel, paclitaxel, derivatives of paclitaxel and cabazitaxel. According to specific embodiments the taxene is docetaxel.

The methods of the present invention are further useful for the treatment of tumor cells which are resistant to conventional chemotherapy. Tumor cell resistance to chemotherapy can be attributed to (a) overexpression of drug efflux pumps, such as P-glycoprotein; (b) acquired mutations at the drug binding site of tubulin; (c) differential expression of tubulin isoforms; (d) alteration in apoptotic mechanisms; (e) activation of growth factor pathways; or (f) other biochemical changes (Deepak Sampath et al. Clin Cancer Res 2006; 12(11):3459-69). The contribution of each of these mechanisms to clinical resistance remains uncertain, although correlations have been made with P-glycoprotein expression levels in some tumor types. It has been surprisingly found that the pharmaceutical compositions disclosed herein can effectively kill chemotherapy resistant tumor cells. Particularly it has been shown that docetaxel sustained release pharmaceutical compositions, as disclosed above, efficiently kill cancer cells resistant to docetaxel. Without wishing to by bound by theory or mechanism of action it is suggested that the combination of high local concentration and prolonged release generates high and prolonged exposure to the drug that effectively overcomes resistant mechanisms based on efflux (MDR) pumps. Non limiting list of tumor cells resistant to chemotherapy include HCT-8 colorectal carcinoma cells (IC₅₀ docetaxel—3070 nM, IC₅₀ paclitaxel 3290 nM), GXF-209 gastric cancer cells, UISO BCA-1 breast cancer cells, P02 pancreas cells, 3LL Lewis lung cancer, KB-8-5 (IC₅₀ docetaxel—8.8 nM, IC₅₀ paclitaxel 70.2 nM), KB-P-15 (IC₅₀ docetaxel—17.6 nM, IC₅₀ paclitaxel 117 nM), KB-D-15 (IC₅₀ docetaxel—68.2 nM, IC₅₀ paclitaxel 565.5 nM), KB-V-1 (IC₅₀ docetaxel—467.5 nM, IC₅₀ paclitaxel 3202 nM) and KB-PTX/099 (IC₅₀ docetaxel—8.8 nM, IC₅₀ paclitaxel 74.1 nM) Epidermoid cells, DLD-1 (IC₅₀ docetaxel—16.2 nM, IC₅₀ paclitaxel 32.8 nM) and HCT-15 (IC₅₀ docetaxel—54.1 nM, IC₅₀ paclitaxel 434.6 nM) colorectal carcinoma cells and A549.EpoB40 non squamous cell lung carcinoma (IC₅₀ docetaxel—28.5 nM, IC₅₀ paclitaxel 127.5 nM). According to some embodiments, the methods of the invention may be suitable to any other chemotherapy resistant tumor, wherein their resistance is a result of overexpression of drug efflux pumps.

The efficacy of a drug is commonly determined by its local concentration in the interstitial fluids around tumor cells. That, in turn, is determined by the ratio between the accumulation rates of drug released from the pharmaceutical composition vs. its elimination (for example, by physical distribution to surrounding tissue). Without being limited by theory or mechanism of action it is suggested that the ability to generate high local concentration of bioavailable taxene drug within the tumor or within the inner surface of a resection site after removal of the tumor by surgery, for a sufficient duration of time, is the major factor in the ability of the pharmaceutical compositions disclosed herein to efficiently kill tumor cells and even tumor cells which are resistant to the drug in use (i.e. treating a docetaxel resistant tumor with a pharmaceutical composition comprising docetaxel). One of the ways to gain better control over the local effect of taxene (e.g. docetaxel) is by controlling: 1) its release profile from the pharmaceutical composition, 2) its' release rate and 3) the duration of its release. These parameters are closely related; while the release rate is strongly depended on the specific formulation (i.e. the ratio between the polymer, lipids and the taxane), the duration is a function of two factors: release rate and the size of drug reservoir (which may be achieved, for example, by changing the ratio between the tri-calcium phosphate particles and the amount of the organic components). It is well known in the art that Increasing the efflux of drugs from the intracellular compartment via energy-dependent efflux pumps is a natural mechanism in cells. This mechanism is also responsible for the development of resistance to chemotherapy. One of the ways to overcome resistant cells, is to overwhelm the efflux pumps with high concentration of the drug over extended periods of time. Thus, it is suggested that as long as the concentration of bioavailable taxane at the tumor site is sufficient, and the duration of exposure of the tumor cells to said taxene is adequate, the taxane will be capable of killing taxane resistant tumor cells.

According to some embodiments the pharmaceutical composition of the invention is in the form of a powder. According to some embodiments, the powder is substantially free of water. According to other embodiments, the powder is a dry powder. According to some embodiments, the powder particle size is dictated by the particle size of the biodegradable mineral substrate. The polymer-lipid matrix which is coating the biodegradable substrate is partly included into the inner space of the porous biodegradable substrate. According to some embodiments, the polymer-lipid the may have an average diameter (as measured by laser diffraction) of at least about 30 μm, at least about 40 μm, at least about 50 μm, at least about 60 μm, at least about 70 μm, at least about 80 μm, at least about 90 μm, at least about 100 μm, between 30 μm and 120 μm, between 30 μm and 100 μm, between 50 μm and 100 μm, not more than about 150 μm, not more than about 140 μm, not more than about 130 μm, not more than about 120 μm, not more than about 110 μm, not more than about 100 μm. Each possibility represents a separate embodiment of the invention. According to some embodiments, the powder is spread or sprinkled over the surface of the tumor or applied to the inner surface of the resection cavity. According to some embodiments, the powder is spread or sprinkled on the surface of the solid tumor or the surface of the resection cavity at an amount ranging from 20 mg to 500 mg per surface area of 1 cm². According to alternative embodiments, the composition is applied at an amount ranging from 50 mg to 400 mg, 50 mg to 350 mg, 50 mg to 300 mg, 50 mg to 275 mg, 50 mg to 250 mg, 50 mg to 225 mg, 50 mg to 200 mg, 50 mg to 180 mg, 50 mg to 170 mg; 50 mg to 160 mg; between 50 mg to 150 mg; between 50 mg to 120 mg; between 50 mg to 100 mg; 50 mg to 100 mg; between 75 mg to 160 mg; between 75 mg to 120 mg; between 75 mg to 100 mg per 1 cm².

According to certain embodiments of the invention, the pharmaceutical composition is formulated as a paste prior to its application to the tumor site or tumor wall of the resected tumor cavity following resection of the tumor. According to some embodiments, the paste is spread over the surface of the tumor or applied to the inner surface of the resection cavity. Typically, a paste like structure is obtained by hydrating the particulate pharmaceutical composition with an aqueous solution prior to its application e.g. saline water (0.9% saline solution). According to some embodiments, hydration shall be performed not more than 2 hours prior to the application of the resulting paste to the tumor site, preferably up to 1 hour prior to the application of the resulting paste to the tumor site, more preferably, not more than 30 minutes prior to its application to the tumor site. According to some embodiments, a paste texture will be attained when the amount of aqueous solution (for example: saline) mixed with the pharmaceutical composition is between 0.1:1 and 1:1 (w/w) respectively; preferably between 0.3:1 and 0.6:1 (w/w) respectively. According to some embodiments, the aqueous solution added to the dry pharmaceutical composition powder for the formation of a paste as described above, does not change the overall volume of the pharmaceutical composition powder being hydrated, therefore leaving the overall volume almost unchanged. According to some embodiments, the paste is spread on the surface of the tumor or the surface of the resection cavity forming a thin and uniform layer having a thickness of up to 5 mm; alternatively, up to 4 mm; alternatively up to 3 mm; preferably between 1 to 3 mm thick.

The pharmaceutical compositions disclosed herein, according to additional embodiments, may be administered intratumorally, typically by injection, generating a neoadjuvant therapy, typically prior to surgery. According to some embodiments, the pharmaceutical composition may be injected directly into the tumor as a dry powder using apparatus suitable for the injection of dry powders (non limiting examples are disclosed in U.S. Pat. No. 8,579,855, however any other suitable medical apparatus known in the art for the delivery of powders may be used). Alternatively, the pharmaceutical composition may be injected as a liquid suspension. Clinically used standard syringes, needles, tubing systems and cannulae may be used for injecting the liquid suspension. The liquid suspension may preferably be prepared such that the minimal amount of a continuous liquid phase is added to the pharmaceutical composition powder suitable for the formation of a suspension for injection. According to some embodiments, a suspension for injection will be attained when the amount of continuous liquid phase (for example: an aqueous phase) mixed with the pharmaceutical composition powder is between 0.1:1 and 2:1 (w/w) respectively; preferably between 0.3:1 and 1:1 (w/w) respectively, more preferably between 0.3:1 and 0.6:1 (w/w) respectively. The volume of the pharmaceutical suspension injected may not exceed 50% of the volume of the solid tumor, preferably less than 45%, less than 40%, less than 35%, less than 30%, less than 25%, less than 20%, less than 15% of the volume of the tumor. Each possibility represents a separate embodiment of the invention. The volume of the suspension may be preferably divided into more than one injection, preferably injected to different parts of the tumor in order to spread the dosage over the whole or substantially the whole volume of the tumor. Due to the inherent properties of the biodegradable particulate substrate contained in the pharmaceutical compositions of the invention, the composition is radio-opaque and observable with standard clinical radioscopy methods, thus the positioning of pharmaceutical compositions disclosed herein can be monitored during injection and during the treatment period by e.g. ultrasound imaging; magnetic resonance imaging; X-ray transmission imaging; computer tomography imaging; isotope based imaging including positron emission tomography or gamma camera/SPECT; magnetic- or radio-wave based positioning systems.

In some embodiments, the suspension for injection can comprise water (e.g. saline) and optionally one or more excipients selected from the group consisting of a buffer, a tonicity adjusting agent, a viscosity modifier, a lubricant, an osmotic agent and a surfactant. For example, the suspension can comprise the pharmaceutical composition particles, water, lubricant. In some embodiments, the suspension consists essentially of or consists of water, the pharmaceutical composition particles suspended in saline and a surfactant. Non limiting example of surfactant that can be used include polysorbates (such as, polysorbate 20, polysorbate 21, polysorbate 40, polysorbate 60, polysorbate 61, polysorbate 65, polysorbate 80, polysorbate 81, polysorbate 85, and polysorbate 120), lauryl sulfates, acetylated monoglycerides, diacetylated monoglycerides, and poloxamers. The suspension can comprise one or more tonicity adjusting agents. Suitable tonicity adjusting agents include by way of example and without limitation, one or more inorganic salts, electrolytes, sodium chloride, potassium chloride, sodium phosphate, potassium phosphate, sodium, potassium sulfates, sodium and potassium bicarbonates and alkaline earth metal salts, such as alkaline earth metal inorganic salts, e.g., calcium salts, and magnesium salts, mannitol, dextrose, glycerin, propylene glycol, and mixtures thereof. The suspension can comprise one or more demulcents. Suitable demulcents include cellulose derivatives such as carboxymethylcellulose sodium, hydroxyethyl cellulose, hydroxypropyl methylcellulose, and methylcellulose; gelatin, glycerin, polyethylene glycol 300, polyethylene glycol 400, and propylene glycol. The suspension can comprise a viscosity modifiers that increase or decrease the viscosity of the suspension. Suitable viscosity modifiers include methylcellulose, hydroxypropyl methylcellulose, mannitol and polyvinylpyrrolidone. The suspension can comprise one or more lubricants. Suitable lubricants include natural and synthetic phospholipids (such as for example DMPC) or hyaluronic acid.

### EXAMPLES

### Example 1—Docetaxel Extended-Release Formulations Comprising Different Phospholipids

Formulations comprising different phosphatidylcholine with and without cholesterol were prepared. The ratio between the formulation components tested were as follows: TCP:(DMPC, DPPC, DSPC or DOPC):PLGA:DTX at a ratio of 1000:90:30:10 and TCP:(DMPC, DPPC, DSPC or DOPC):PLGA:CH:DTX at a ratio of 1000:90:30:15:10.

The formulations were prepared according to the following exemplary protocol:
a) Into eight 5 ml volumetric flasks PLGA (100 mg), CH (were needed 50 mg), docetaxel (33.3 mg) and phosphatidylcholine (300 mg) were added followed by the addition of EA:EtOH mixture to dissolve the solids.
b) Whenever needed, the mixtures were heated to 40° C.-45° C. to help dissolve the phospholipids.
c) 1.5 g of β-TCP particles (50-100 μm) were added to each of eight 30 mm petri dishes and 2.25 mL of the 8 organic solutions prepared in step (a) were added on top of the TCP.
d) The petri dishes were left uncovered on a dry heating block set to 45° C. for about 45 minutes and were than covered and put under vacuum (at room temperature) over-night for complete solvent evaporation.
e) All 8 formulation were transferred to 20 ml scintillation vials and kept at 4° C. protected from light.

Docetaxel release—250 mg of each of the tested formulations were put in a 20 ml vial to which 5 ml of PBS were added slowly and the samples were placed in an incubator at 37° C. Once a day the PBS medium was collected and analyzed. 5 ml of fresh PBS was then added to the vials. The released drug concentration was quantified using HPLC. Release analysis was stopped after 13 days. The formulation remains were left to dry over-night in vacuum at RT. The amount of docetaxel and its' 7-Epi impurity in the formulation remains were quantified.

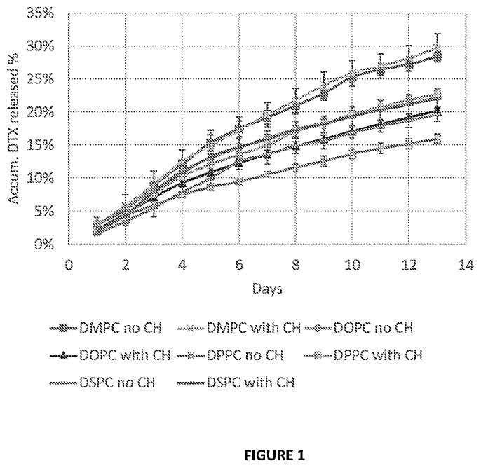

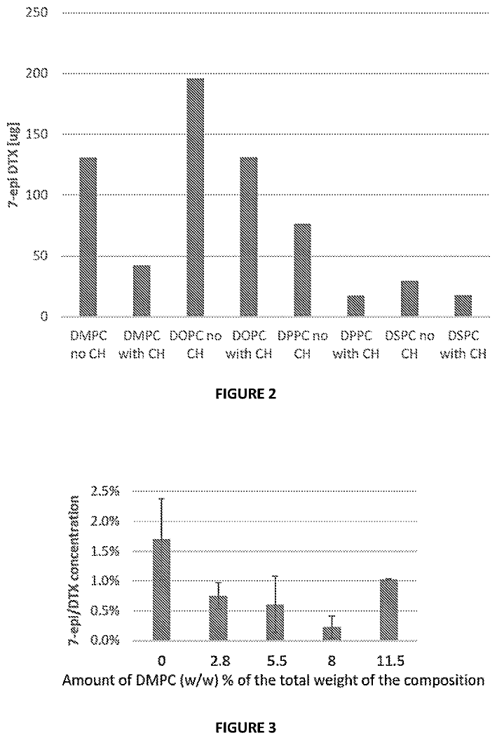

As can be seen in FIG. 1, the docetaxel is released faster and more efficiently from compositions comprising DMPC as compared to its release from similar compositions comprising phospholipids having longer hydrocarbon chains and higher phase transition temperatures (e.g. DPPC and DSPC). Compositions comprising phospholipids with saturated hydrocarbon chains longer than 14 carbons did not reach the full release potential within 6 weeks, which is typically the limited time window between tumor resection surgeries and further adjuvant treatments including radiation or systemic chemo typically given as preventive treatment post tumor resection. Furthermore, it has been shown that compositions comprising cholesterol better protected the docetaxel reservoir from transforming to its 7-epimer than similar compositions without cholesterol (FIG. 2).

### Example 2—Docetaxel Extended-Release Formulations Comprising Different Amounts of DMPC

Materials

PLGA (Corbion, Purac 7502); docetaxel (DTX) (TAPI); DMPC (Lipoid); TCP (Cam bioceramics, 50-100 μm)

The ratio between the formulation ingredients TCP:DMPC:PLGA:DTX was 1000:(0,30,60,90,135):30:10 respectively, which is equivalent to 0, 2.8%, 5.5%, 8% and 11.5% (w/w) of DMPC from the total weight of the formulation. Formulations were prepared and the release of docetaxel from the formulations was performed as described above in Example 1.

As can be seen in FIG. 3, the relative 7-Epi content was found to be the highest in DMPC free formulations and its relative amount was greatly reduced in formulations comprising DMPC.

### Example 3—Docetaxel Extended-Release Formulations Comprising Detergents

Formulations comprising the detergent Tween 80 have been prepared and the release profile of said formulations were generated as described above in example 1.

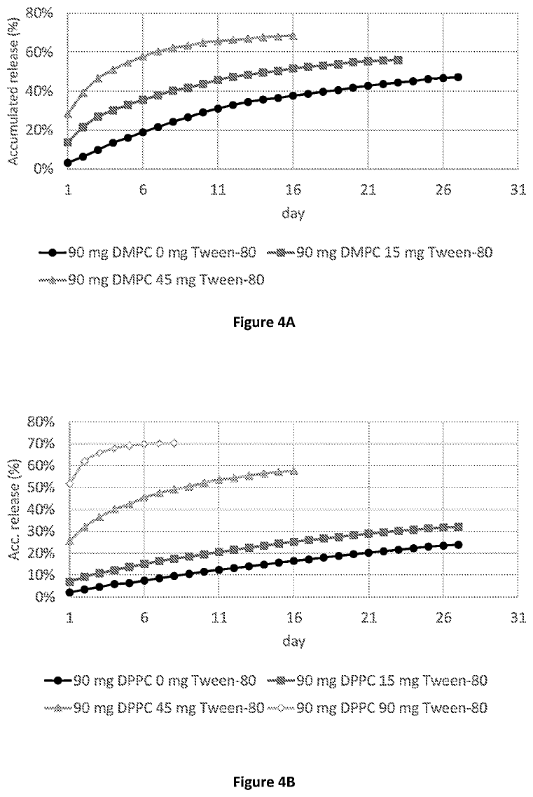

Formulation comprising either DMPC or DPPC as the lipid component and further comprising Tween 80 have been prepared. The ratio between the formulation ingredients TCP:DMPC:PLGA:DTX:Tween-80 was 1000:90:30:10:(0,15,45) respectively (FIG. 4A). Formulation comprising DPPC as the lipid component were prepared wherein the ratio between the formulation ingredients was TCP:DMPC:PLGA:DTX:Tween-80 was 1000:90:30:10:(0,15,45,90) respectively (FIG. 4B).

FIGS. 4A and 4B show that the addition of Tween-80 to the sustained release composition, increased the release rate however, it influenced the overall release profile which, in the presence of Tween-80 was characterized by an unwanted burst release, that may lead to significant local and systemic toxicity.

### Example 4—Docetaxel Sustained Release Formulation with Varying Amounts of Cholesterol

Formulations comprising different amounts of cholesterol (CH) have been prepared.

The ratio between the formulation components tested were as follows: TCP:DMPC:PLGA:DTX:CH at a ratio of 1000:90:30:10:(0,15,30), equivalent to formulation with 0, 1.3% and 2.6% of cholesterol (w/w) from the total weight of the formulation

It has been found that docetaxel transformation to its' 7-epimer has been reduced in formulations comprising cholesterol (FIG. 1). Furthermore, it was found that the addition of cholesterol was effective in protecting docetaxel during storage (see table 2).

FIG. 5 shows that the higher the cholesterol concentration the lower the percentage of the 7-epimer of docetaxel in the formulation. However, due to cholesterol limited solubility in the preparation mixture, a concentration lower than 2.6% of cholesterol w/w of the total weight of the formulation should preferably be used.

Table 1 lists additional formulations comprising various TCP/DMPC/PLGA/Cholesterol/DTX in which formulations with or without cholesterol are compared.

TABLE 1
Docetaxel sustained release formulations according to certain
embodiments of the invention

| | DMPC | PLGA | CH | DTX | TCP | % DTX |
|---|---|---|---|---|---|---|
| | [mg] | [mg] | [mg] | [mg] | [mg] | (w/w) |
| Formulation I | 90 | 30 | − | 10 | 1000 | 0.885 |
| Formulation II | 90 | 30 | 15 | 10 | 1000 | 0.873 |
| Formulation III | 45 | 15 | − | 6 | 1000 | 0.563 |
| Formulation IV | 45 | 15 | 7.5 | 6 | 1000 | 0.559 |

Table 2 summarizes the results of a stability assay performed with the formulations I-IV listed in table 1 showing that the presence of cholesterol reduced and even stopped completely the formation of 7-epimer of docetaxel in the formulation.

TABLE 2
stability assay of various formulations according
to certain embodiments of the invention

| | | | 7-epi | 7-epi | 7-epi |
|---|---|---|---|---|---|
| Formulation | Storage | | DTX/DTX | DTX/DTX | DTX/DTX |
| No. | temperature | | at t = 0 | at t = 4 weeks | at t = 9 weeks |
| I | 4° | C. | 0.41% | 0 | 0 |
| | RT | | 0.41% | 0.57% | 0.83% |
| | 37° | C. | 0.41% | 0.41% | 0.45% |
| II | 4° | C. | 0 | 0 | 0 |
| | RT | | 0 | 0 | 0 |
| | 37° | C. | 0 | 0.36% | 0.32% |
| III | 4° | C. | 0.4% | 0.06% | 0.38% |
| | RT | | 0.4% | 1.44% | 2.14% |
| | 37° | C. | 0.4% | 2.05% | 1.98% |
| IV | 4° | C. | 0 | 0 | 0 |
| | RT | | 0 | 0 | 0.29% |
| | 37° | C. | 0 | 1.37% | 1.48% |

According to an embodiment of the present invention the presence of cholesterol in the sustained release composition of docetaxel chemically stabilized the docetaxel and results in a composition with a content of 7-epi-docetaxel below 0.5% after being stored for 9 weeks (e.g. at room temperature). Particularly, the content of 7-epi-docetaxel is preferably below 0.4%, such as about 0.35%, about 0.3%, about 0.25%, about 0.20% or even lower, after 9 weeks of storage at room temperature.

The term “chemically stable” means that the chemical structure docetaxel is stable when the pharmaceutical composition of the present invention is stored under conventional conditions.

Preferably, after storage at 2-8° C. for at least 24 months, the content percentage of 7-epi-docetaxel is less than 1% preferably, less than 0.5%.

### Example 5—Sustained Release Paclitaxel Formulations

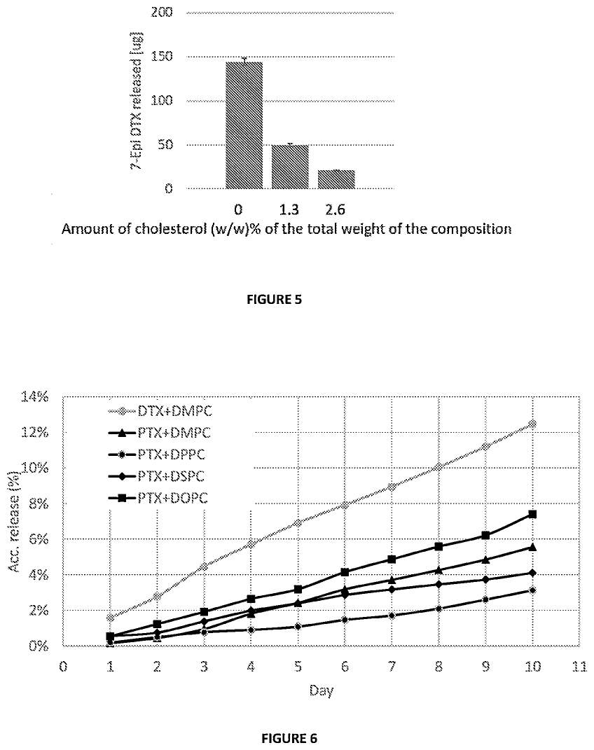

Paclitaxel (PTX) sustained release compositions have been prepared as described above in example 1. The ratio between the formulation components tested were as follows: TCP:(DMPC, DPPC, DSPC or DOPC):PLGA:CH:PTX at a ratio of 1000:90:30:15:10. The release of paclitaxel from the composition was followed as described above in Example 1 and the zero-order release profile is presented in FIG. 6.

### Example 6—Sustained Release Docetaxel Formulations Comprising Poly-Ethylene Glycol (PEG)

A formulation comprising PEG 4000 as the polymer was prepared as described in Example 1. The ratio between the formulation components TCP:DMPC:PEG:Cholesterol:docetaxel was as follows: 1000:90:30:15:10.

The release of docetaxel from the formulation comprising PEG 4000 has been followed using the dissolution analysis (USP1 dissolution apparatus—Sotax AT7 smart with baskets at 50 RPM) and was compared to the release of docetaxel from a similar formulation comprising PLGA as the polymer.

1 g of formulation was dissolved in 0.5% SDS in PBS (phosphate buffered saline), 500 ml of medium in each vessel. Sampling timepoints—1 h, 2 h, 4 h, 6 h, 24 h.

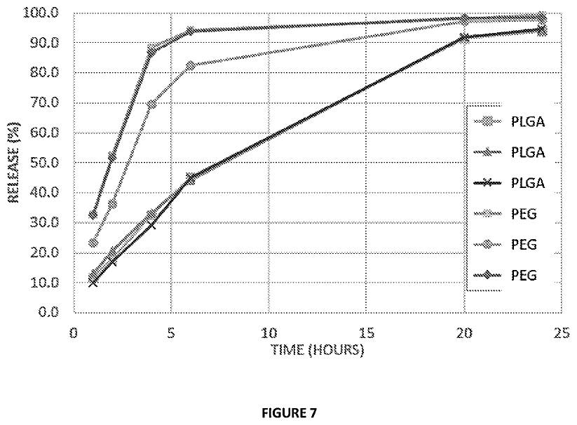

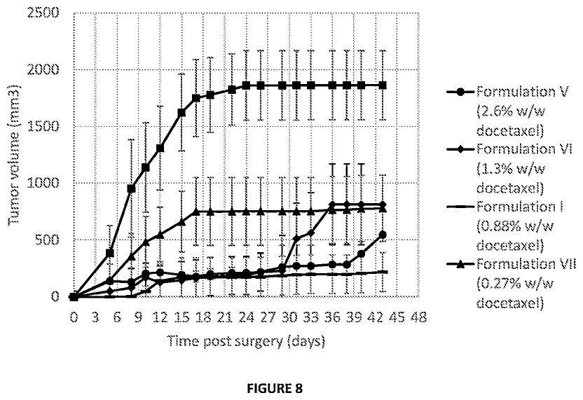

As shown in FIG. 7, the presence of PEG 4000 resulted with a burst release of the encapsulated docetaxel with more than 90% of the drug being released within 5 hours. In comparison, the release of docetaxel from the formulation comprising PLGA was greatly extended and displayed prolonged zero order kinetics with 90% of the drug being released within 20 hours.

### Example 7—Evaluation of the Antitumor Effect of Pharmaceutical Compositions According to Some Embodiments of the Invention Comprising Different Amounts of Docetaxel (DTX) on the Recurrence of CT26 Cell Line in Mouse Syngeneic Tumor Model, In-Vivo

This study was performed to assess the antitumor effect of sustained release formulations according to exemplary embodiments of the invention with different docetaxel doses on CT26 colon carcinoma cell line tumors in BALB/c mice. (7-8 weeks old, weighing 16-20+/− gram at study initiation).

Tested Formulations:

PLEX-DTX containing 2.6% Docetaxel (TCP:DMPC:PLGA:DTX (w/w) equals 1000:90:30:30) Formulation V

PLEX-DTX containing 1.3% Docetaxel (TCP:DMPC:PLGA:DTX (w/w) equals 1000:90:30:15) Formulation VI

PLEX-DTX containing 0.88% Docetaxel (TCP:DMPC:PLGA:DTX (w/w) equals 1000:90:30:10) Formulation I

PLEX-DTX containing 0.27% Docetaxel (TCP:DMPC:PLGA:DTX (w/w) equals 1000:90:30:3) Formulation VII

Control: Saline

Disease induction: Transplantation of CT-26 subcutaneous tumor, a cell line resistant to decetaxel (IC₅₀ 260 nM). For the purpose of comparison, the IC₅₀ of cell lines which are non-resistant to docetaxel are in the range of few nM, Examples include NSCLC: A549 cells (1.9 nM),CRC: HCT-116 cells (5.4 nM) and epidermoid KB-3-1 cells (1.1 nM) [Preclinical Pharmacologic Evaluation of MST-997, an Orally Active Taxane with Superior In vitro and In vivo Efficacy in Paclitaxel- and Docetaxel-Resistant Tumor Models (Clin Cancer Res 2006, 12:3459-69)]

Mice were injected SC with 0.5 million CT-26 cells above the right hip. After 11 days, the tumors reached the desired volume (˜400 mm³ the animals were divided to five groups, mice were anesthetized, and the tumors were resected. Groups 1-4 were SC administered the test formulations (200 mg) in the tumor bed, each group with a formulation containing different concentrations of docetaxel (2.6%, 1.3%, 0.88% or 0.27% w/w (Table 3)), or saline was given locally (Group 5). The skin incision was then closed using a sterile suture. Post-surgery the animals were returned to their cages for recovery and observation. Tumor size, clinical signs, and body weights were followed for 43 days.

TABLE 3
Study design, group designation

| | | | Treatment | Docetaxel amount |
|---|---|---|---|---|
| Group | # mice | Test-item | mg/animal | (mg/mouse) |
| 1 | 8 | Formulation V | 200 | 5.2 |
| 2 | 9 | Formulation VI | 200 | 2.6 |
| 3 | 9 | Formulation I | 200 | 1.73 |
| 4 | 8 | Formulation VII | 200 | 0.52 |
| 5 | 8 | untreated | saline | NA |

Results

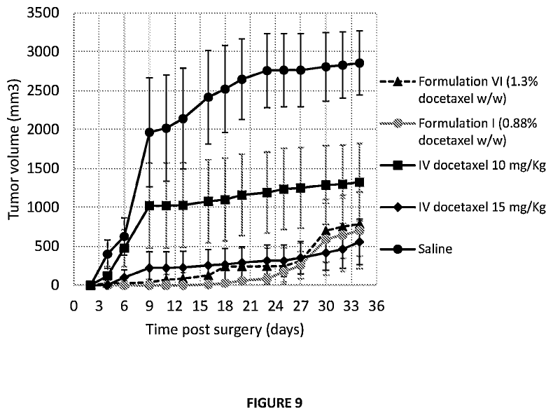

At the end of the study (Day 43), the number of tumor free animals varied between the DTX-treated groups. At the highest docetaxel dose (5.2 mg/mouse), 4/8 animals were tumor free; in Group 2 (2.6 mg/mouse), 5/9 animals were tumor free; in Group 3 (1.73 mg/mouse), 7/9 animals were tumor free; and, in Group 4 (0.52 mg/mouse), 3/8 animals were tumor free. No tumor free animals were observed in Group 5. The average tumor volume was significantly smaller (p<0.05) in the DTX treated groups (548 mm³, 814 mm³, 218 mm³ and 872 mm³ for Group 1, Group 2, Group 3, and Group 4, respectively; FIG. 8) than in the saline-treated group (Group 5; 2091 mm³). The large standard deviation within the groups reflects the large variability in tumor size within the group.

The survival rate was 63% (5/8), 56% (5/9), 90% (8/9), and 50% (4/8) in Groups 1, 2, 3, and 4, respectively and 0% (0/8) in Group 5 (untreated). In Group 1 (2.6% docetaxel), 2 animals were humanely sacrificed due to severe weight loss (Day 19) and 1 animal was sacrificed due to tumor volume that exceeded 1500 mm³ (Day 43). In Group 2 (1.3% docetaxel), 3 animals were sacrificed due to tumor volume that exceeded 1500 mm³ (Days 22, 31 and 36), and 1 animal was found dead (Day 36). In Group 3 (0.88% docetaxel), only 1 animal was terminated early due to tumor volume that exceeded 1500 mm³ (Day 15). In Group 4 (0.27% docetaxel), 4 animals were terminated early due to tumor volume that exceeded 1500 mm³ (Days 10, 12, and 17). In Group 5 (untreated) all animals were sacrificed due to tumor volume that exceeded 1500 mm³ by Day 24. While in the saline-control group all the animals were terminated by Day 24, in the groups treated with the docetaxel formulations, according to some embodiments of the invention, most of the animals survived until study termination (Day 43).

Body Weight—To reduce the impact of the tumor weight on the animals' total body weight, a calibration curve plotting the actual tumor weight vs. tumor volume was made based on the resected tumors. This plot enabled an estimation of the tumor weight based on its volume and this tumor weight was subtracted from the actual weight of the tumor bearing animals, thus enabling a measurement of animal weight during the study follow-up. Animal weight was measured three-times a week during the course of the study. The weight was normalized to the weight of the animals on the day of tumor resection and treatment initiation.

Animals in Groups 1 and 2 (2.6% docetaxel and 1.3% docetaxel, respectively) suffered from weight loss with maximal decrease of 20% and 9%, respectively on Day 17. In both Groups 1 and 2 animals, weight gain was observed after Day 17; by study termination, these animals weighed 115-116% of their original weight. Animals in Group 3 (0.88% docetaxel) had minor weight loss (˜2%) up to two weeks post administration, but weight gain was observed on Day 17 and thereafter, reaching 113% of their original weight by study termination. Animals in Group 4 (0.27% docetaxel) and the untreated group (Group 5) started to gain weight at Day 3 post-surgery.

Discussion—The anti-tumor effect of the treatment with various docetaxel formulations according to some exemplary embodiments of the invention, each with different concentration of docetaxel, was demonstrated compared to a saline-treated group. All formulations increased animal survival compared to the saline-treated group. However, symptoms that are related to docetaxel toxicity were more frequent in the docetaxel formulations with the higher concentration of docetaxel (1.3% docetaxel (Formulation VI) and 2.6% docetaxel (Formulation V)).

Interestingly the formulation with a lower docetaxel concentration (0.88% (Formulation I); 1.76 mg/mouse) showed minimal weight loss and was concluded to be safer. This dose was also more effective in decreasing tumor reoccurrence in mice than the formulation with the lowest docetaxel concentration (0.27% (Formulation VII); 0.54 mg/mouse).

### Example 8—Evaluation of the Antitumor Effect of the Formulations According to Embodiments of the Invention on Mouse Syngeneic Tumor Model

In the current experiment, the efficacy of local treatment with extended-release formulations according to some embodiments of the invention was compared to systemic treatment with Docetaxel. For that, subcutaneous colon carcinoma tumors were established in female BALB/c mice (7-8 weeks old, weighing ±16-20 gram at study initiation) and after reaching a desired volume (400-600 mm³), they were resected and ˜90% of their volume was removed followed by administration of the test items. The recurrence rate of the tumors was followed and compared to an untreated control group.

Study Design:

Animals were injected SC with 0.5 million CT-26 cells above the right hip. When the tumor reached the desired volume (400 mm³) after ˜7 days, animals were divided to five groups, mice were anesthetized, and the tumors were resected. Group 1 was administered with 200 mg of formulation VI containing 1.3% docetaxel (2.6 mg/mouse) to the tumor bed and Group 2 was administered with 200 mg of formulation I containing 0.88% docetaxel (1.72 mg/mouse) to the tumor bed. Groups 3 and 4 were treated by repeated i.v injections of docetaxel solution. Group 3 was administered with 20 mg/kg i.v followed by five i.v injections of 10 mg/kg, once every 4 days. Group 4 was administered with 30 mg/kg i.v followed by five IV injections of 15 mg/kg, once every 4 days. Group 5 served as a saline-treated control with ˜100 μL saline administered locally into the tumor bed. The skin incision was then closed using a sterile suture. Post-surgery, the animals were returned to their cages for recovery and observation. Tumor size, clinical signs, and body weights were followed for 39 days. The full study design is presented in Table 4.

TABLE 4
Example 8 group designation

| | | | | Docetaxel | |
|---|---|---|---|---|---|
| | | | | amount | Adminis- |
| | # | | | (mg/ | tration |
| Group | mice | Test-item | Treatment | mouse) | route |
| 1 | 8 | Formulation VI | 200 mg/ | 2.6 | local SC |
| | | containing 1.3% | animal | | |
| | | Docetaxel (w/w) | | | |
| 2 | 8 | Formulation I | 200 mg/ | 1.72 | local SC |
| | | containing 0.88% | animal | | |
| | | Docetaxel (w/w) | | | |
| 3 | 8 | Docetaxel | 10 mg/kg | 1.75 | repeated |
| | | | every 4 days | | iv (x5) |
| 4 | 8 | Docetaxel | 15 mg/kg | 2.6 | repeated |
| | | | every 4 days | | iv (x5) |
| 5 | 8 | Saline | 100 μL | NA | local SC |

### Experimental Procedures

Study Results

At the end of the study (Day 39), 5/8 animals were tumor free in Group 1. In Group 2, 6/8 animals were tumor free. In Group 3 (i.v docetaxel), 2/8 animals were tumor free. In Group 4 (i.v docetaxel), 3/8 animals were tumor free. In Group 5 (saline-treated), all animals had tumors.

After 39 days, the average tumor volume was significantly smaller (p<0.05) in the treated groups 1-4 (563 mm³, 375 mm³, 955 mm³ and 485 mm³ for Group 1, Group 2, Group 3, and Group 4, respectively, FIG. 9) than in the saline control group (1500 mm³). The large standard deviation within the groups reflects the large variability in tumor size within the group.

The survival rate in the groups treated with the sustained release formulation according to embodiments of the invention was 63% (5/8) and 75% (6/8) in Groups 1 and 2,respectively. In the IV docetaxel-treated groups, the survival rate was 50% (4/8) and 63% (5/8 in Groups 3 and 4, respectively. In Group 5 (saline-control) the survival rate was only 12.5% (1/8). In Group 1 (formulation VI, 1.3% docetaxel), 3 animals were sacrificed early due to tumor volume that exceeded 1500 mm³ (Days 18, 30 and 37). In Group 2 (formulation I, 0.88% docetaxel), 2 animals were sacrificed early due to tumor volume that exceeded 1500 mm³ (Days 30 and 34). In Group 3 (i.v docetaxel 10 mg/kg), 4 animals were terminated early due to tumor volume that exceeded 1500 mm³ (3 animals on Day 10 and one on Day 25). In Group 4 (i.v docetaxel 15 mg/kg), 1 animal was terminated early due to severe weight loss and bad physical condition (Day 20) and 2 animals were terminated early due to tumor volume that exceeded 1500 mm³ (Days 10 and 34). In the saline-control group, 8 animals were sacrificed due to tumor volume that exceeded 1500 mm³ (4 animals on Day 10, and 1 each on Days 16, 20, 23, and 37).

Animal weight was measured three-times a week during the study as described above in Example 5. Animals in groups 1, 2, 3 and 4 suffered from weight loss with maximal decreases of 12% (Day 16), 8% (Day 16), 8% (Day 16) and 17% (Day 20), respectively. Animals in Group 5 (saline-control) did not show weight loss due to early tumor development that increased the mice weight. Overall, the groups treated with the sustained release formulations disclosed herein and i.v docetaxel treatment groups started to gain weight on Days 18, 18, 20 and 23 (for Groups 1, 2, 3 and 4, respectively).

Conclusions: Local application of both Formulation I and Formulation VI displayed high efficacy in decreasing tumor recurrence and increasing overall survival. Both formulations showed similar efficacy. The systemic Docetaxel treatment of 15 mg/Kg (2.6 mg/mouse total dose) displayed lower efficacy in terms of tumor free survival rate versus the local treatment pointing to the superiority of the local treatment. Additionally, the systemic treatment caused severe systemic toxicity reflected in the animal weight loss. Weight loss was less pronounced in group 2 (Formulation I, 0.88% docetaxel), although the exposure to the overall dose of docetaxel administered in both groups was similar (˜1.7 mg).

### Example 9—Evaluation of the Antitumor Effect of Extended-Release Formulation According to Exemplary Embodiments of the Invention on U87 GBM Cell Line In Vivo Mouse Xenograft Tumor Model

This study was performed to assess the efficacy of the anti-tumor effect of different amounts of the sustained release compositions according to some exemplary embodiments of the invention on U87 human GBM cell line tumor xenograft in nude mice.

Study Design

Mice were injected subcutaneously (SC) with 3 million U87 cells above the right hip. When the tumor reached the volume of about 400 mm³ after about 9 days, animals were divided to six groups (n=10/group), the mice were anesthetized, and the tumors were resected. The tumor bed sizes were measured and documented. Groups 1, 2 and 3 were administered 20, 50, or 100 mg of formulation II; 0.87% docetaxel locally on the tumor bed, respectively. Group 4 was administered 100 mg of formulation II vehicle (excipients only without DTX) locally on the tumor bed. Group 5 served as a saline control in which ˜100 μL saline was administered locally into the tumor bed. Group 6 served as positive control and was treated with gemcitabine (300 mg/kg administered as an intraperitoneal injection, four times, every 7 days). The skin incision was then closed using a sterile suture. Post-surgery, the animals were returned to their cages for recovery and observation. Tumor size, clinical signs, and body weights were followed for 43 days.

Study Results

After tumor resection, the area of the tumor bed was measured. The average area of the tumor bed was 134±17 mm. The applications of Formulation II were calculated and normalized to amount per 1 cm² tumor bed area. The normalized application rates and docetaxel doses are detailed in Table 5.

TABLE 5
Formulation II Amounts Applied (mg/cm²)

| | | Tumor | Amount | Docetaxel |
|---|---|---|---|---|
| | OncoPLEX | bed area | Applied | Dose |
| Group | (mg) | (cm²) | (mg/cm²) | (mg/cm²) |
| 1 | 100 | 134 | 75 | 0.65 |
| 2 | 50 | | 37 | 0.33 |
| 3 | 20 | | 15 | 0.13 |

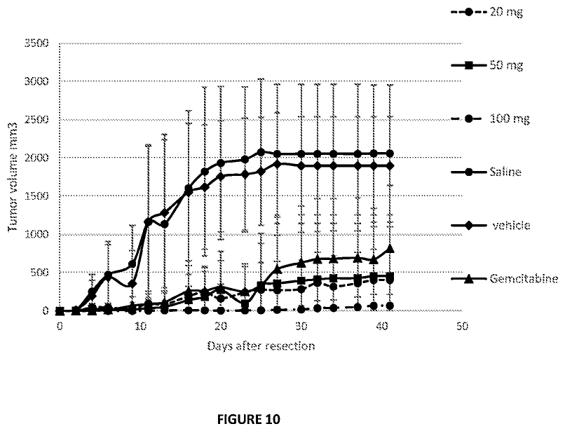

At the end of the study (Day 43), the number of tumor free animals varied between the Formulation II-treated groups. In Group 1 (100 mg of Formulation II), 2/10 animals were tumor free, in Group 2 (50 mg of Formulation II), 1/10 animals were tumor free, and in Group 3 (20 mg of Formulation II), 4/10 animals were tumor free. In Group 4 (100 mg Formulation II vehicle) and in Group 5 (saline control), all animals had tumors. In Group 6 (gemcitabine), 2/10 animals were tumor free. After 43 days (FIG. 10), the average tumor volume was significantly smaller (p<0.001) in all Formulation II- and gemcitabine-treated groups (69 mm³, 456 mm³, 403 mm³ and 780 mm³ for Groups 1, 2, 3, and 6, respectively) than in the Formulation II vehicle- and saline-treated groups (1898 mm³ and 2059 mm³ for Groups 4 and 5, respectively).

The survival rate in Groups 1, 2 and 3 (administered 100, 50, or 20 mg of Formulation II, respectively) was 60% (6/10), 30% (3/10), and 50% (5/10), respectively. In Group 4 (100 mg Formulation II vehicle), only 10% (1/10) survival was recorded. In Group 5 (saline control), no surviving animals were recorded by Day 31. In Group 6 (gemcitabine), the survival rate was 20% (2/10). In Group 1 (100 mg Formulation II), 4 animals were found dead (1 each on Days 20 and 33 and 2 on Day 34). In Group 2 (50 mg Formulation II), 6 animals were found dead (1 each on Days 9, 18, 23, 25, 33 and 39). One (1) animal was terminated early due to tumor volume that exceeded 1500 mm3 on Day 23. In Group 3 (20 mg Formulation II), 5 animals were found dead (1 each on Days 9, 18, 23, 25, 33, and 39). The reason for their death was probably due to systemic toxicity since all these animals showed weight loss of ˜20% in the day before they were found dead. In Group 4 (100 mg Formulation II vehicle), 9 animals were terminated early due to tumor volume that exceeded 1500 mm³ (2 on day 9, 3 on day 13, 3 on day 18 and 1 on day 25). In Group 5 (saline control), 2 animals were found dead (1 each on Days 13 and 23). The reason for their death was unknown. Eight (8) animals were terminated early due to tumor volume that exceeded 1500 mm³ (3 on day 9, 2 on day 13, 1 on day 17, 1 on day 27 and 1 on day 30). In Group 6 (gemcitabine), 4 animals were found dead (1 each on Days 30 and 41 and 2 on Day 34). Four (4) animals were terminated due to tumor volume that exceeded 1500 mm³ (1 each on Days 23, 27, 30, and 33). The reason for the death of most animals in the treated groups (1, 2, 3 and 6) was probably due to systemic toxicity (all these animals showed weight loss of ˜20% in the day before they were found dead).

Animals in Groups 1 and 2 receiving Formulation II (100 or 50 mg, respectively) suffered from weight loss with maximal average decreases of 9% (Day 34) and 2% (Day 13), respectively. Animals in Group 3 (20 mg of Formulation II) did not suffer from weight loss. Animals in Group 4 (Formulation V vehicle) had maximal average weight loss of 2% (Day 6). Animals in Group 5 (saline control) had maximal average weight loss of 5% (Day 23). Animals in Group 6 (gemcitabine) had maximal average weight loss of 13% (Day 34). From the maximal weight loss time point animals in all groups started to gain weight. At Day 43, the weight of the animals in Formulation II treated groups was 99%, 100.5% and 105% of their weight at study initiation for Groups 1, 2, and 3, respectively. In the saline control group (Group 4), Formulation II vehicle group (Group 5), and gemcitabine group (Group 6), the number of the surviving animals was too small to calculate a significant average.

Conclusions

The anti-tumor effect of the treatment with different Formulation II amounts (as reflected in mg/cm²) was demonstrated compared to a saline-control and Formulation II vehicle-treated groups. All Formulation II treatment levels increased animal survival compared to the saline-control group. The groups that were treated with 20 or 50 mg Formulation II (15 or 37 mg/cm²) respectively, decreased the average tumor volume from 1898 mm³ in the saline control group to 403 mm³, and 456 mm³ respectively. The 100 mg Formulation II per animal (75 mg/cm²) treatment had the maximal effect on human GBM tumor recurrence after surgical resection as reflected in the highest number of surviving animals and the lowest overall average tumor volume (69 mm³).

### Example 10—Evaluation of the Antitumor Effect of Extended-Release Formulations According to Embodiments of the Invention on Syngeneic 9L GBM Cell Line Tumor in the Brain of Fischer Rats

This study was performed to assess the anti-tumor effect of different amounts of the sustained release formulation according to some exemplary embodiments of the invention on the survival of animals after induction of syngeneic intra-brain tumors in Fischer rats.

Study Design

Seventy-five (75) animals were designated for this study. The animals were divided into nine groups as described in Table 6. Group 1 served as untreated control. Groups 2 and 3 served as positive controls and were treated with temozolomide (SOC chemotherapy treatment in GBM patients) by gavage in low (33.5 mg/kg) and high doses (50 mg/kg), respectively. Group 4 (n=10) was treated with Formulation II vehicle at the defect site in the same amount as the Formulation II high amount group. Groups 5-8 (n=10/group) were treated at the excision site Formulation II at 5, 10, 25, or 50 mg/defect site. At study initiation, an incision was made and the calvarium bone of all animals was exposed and 5 mm diameter defect was drilled in the calvarium bone. The dura was cut and the brain was exposed. Each animal was injected with 9L cells (105 cells/2 μL/animal) at a depth of ˜1 mm in the brain using a stereotaxic instrument. Following injection of the cells, the incision was sutured. Animals were returned to their cages to recover. Treatment (temozolomide or Formulation II) was set to start five days post cell injection. For Formulation II/Formulation II vehicle treatment, the brain defects in Groups 4-8 were reopened and test articles were administered on top of the site of injection, inside the defect at Day 5. The defects were sealed with bone wax. Animals were returned to their cages to recover. Survival, clinical signs, body weight, and evaluation of cognitive behavior were followed during the study.

TABLE 6
study design

| | | # | Method of | Formulation II | Decetaxel |
|---|---|---|---|---|---|
| Group | Treatment | rats | application | (mg/cm²) | (mg/cm²) |
| 1 | Untreated | 9 | NA | NA | NA |
| 2 | Temozolomide | 8 | PO daily for 5 days | NA | NA |
| | (33.5 mg/kg) | | (equivalent to human | | |
| | | | dose of 200 mg/m²) | | |
| 3 | Temozolomide | 8 | PO daily for 5 days | NA | NA |
| | (50 mg/kg) | | | | |
| 4 | Formulation II vehicle | 10 | Topical defect site | ~255 (vehicle) | NA |
| | (50 mg) | | | | |
| 5 | Formulation II (50 mg) | 10 | Topical defect site | ~255 | 2.2 |
| 6 | Formulation II (25 mg) | 10 | Topical defect site | ~125 | 1.1 |
| 7 | Formulation II (10 mg) | 10 | Topical defect site | ~50 | 0.44 |
| 8 | Formulation II (5 mg) | 10 | Topical defect site | ~25 | 0.22 |

Study results—all the animals had died within five weeks post treatment. In Group 1, the mean survival was 15.8±1.9 days. In Group 2 (temozolomide 33.5 mg/kg), the mean survival was 18.8±2.7 days. In Group 3 (temozolomide 50 mg/kg), the mean survival was 21.8±3.3 days. In Group 4 (Formulation II vehicle), the mean survival was 17.9±2.2 days. In Group 5 (Formulation II 50 mg/animal), the mean survival was 22.8±5.8 days. In Group 6 (Formulation II 25 mg/animal), the mean survival was 20.9±6.5 days. In Group 7 (Formulation II 10 mg/animal), the mean survival was 20.4±4.9 days. In Group 8 (Formulation II 5 mg/animal), the mean survival was 20.4±3.2 days.

Conclusions

Formulation II IC administration five days post tumor cell injection into the brain improved the animal survival at all the tested doses. The anti-tumor effect increased with the amount of Formulation II administered. The strongest effect was achieved at the highest amount of 50 mg Formulation II (0.87% docetaxel w/w) per site (defect diameter 5 mm, defect area 0.196 cm² corresponding to overall 255 mg/cm² of Formulation II (2.2 mg/cm² docetaxel).

### Example 11—Evaluation of Pharmacokinetic (PK) Profile of Locally Administered Docetaxel (DTX) Sustained Release Compositions According to Exemplary Embodiments of the Invention in Rats

This study compared the pharmacokinetics profile of docetaxel sustained release composition according to several embodiments of the invention administered to rats. The systemic PK profile of docetaxel released from the local administered formulations was compared to the PK profile of docetaxel administered i.v.

Animals: 30 Sprague-Dawley female Rats weighing+/−200 gram

Experimental Design—three study groups (n=10) were included in this study. Animals were anesthetized and skin above the right hip was cut (1 cm long) and lifted, creating a SC pocket. By making this pocket, the underlying muscle was slightly injured, mimicking the situation of the resection of SC tumor graft in the rat model. Each animal received the treatment as detailed in Table 7. In Groups 1 and 2, Formulations VI and I respectively were administered in the SC pocket on top of the injured muscle. The skin was then sutured. In the i.v. treated group (Group 3), the administration of treatment was given once, immediately after wound closure. After administration, blood samples were collected in the designated time points. Each treatment group was divided to two subgroups (n=5/subgroup) and each subgroup was sampled at the different time points. Blood was collected at 0.5, 1, 2, 4, 8, 12, and 24 hours and at 2, 3, 4, 5, 6, 7, 14, 21, and 30 days post-administration. Clinical signs and animal weights were followed during the study. The concentration of the released docetaxel in the plasma samples was evaluated by a liquid chromatography tandem mass spectrometry (LC-MS/MS) method (lower limit of quantitation [LLOQ]=3 ng/mL). The results were used to determine the PK profile of docetaxel.

TABLE 7
Study design

| | | | Treat- | Dece- | |
|---|---|---|---|---|---|
| | # | | ment | taxel | |
| | of | | mg/ | amount | Administration |
| Group | rats | Test item | animal | (mg/rat) | route |
| 1 | 10 | Formulation VI | 200 | 2.6 | Local paste (SC) |
| 2 | 10 | Formulation I | 200 | 1.76 | Local paste (SC) |
| 3 | 10 | Docetaxel | 10 mg/kg | 2 | i.v |

Study Results

PK analysis of the plasma samples showed that, the overall exposure in Formulations VI and I was longer than the single i.v administration (Tₗₐₛₜ of 168, 120 and 72 hours for Formulation VI, Formulation I and i.v, respectively; Table 6). The released docetaxel exposure time was correlated to the dose of docetaxel in the extended-release formulation (VI and I). The higher docetaxel dose (1.3%) had longer plasma exposure than the lower docetaxel dose (0.88%). The same trend was observed in the AUC, Cₘₐₓ and t_{1/2}. The Cₘₐₓ of the i.v formulation was more than 10-fold higher than the max exposure of Formulation VI (881 vs. 80.4 ng/ml, Group 3 and Group 1, respectively; Table 4). Since the overall dose of docetaxel in Formulation I and the i.v administration were similar (1.76 and 2 mg/animal, respectively) the AUC values of the two groups were similar as well (2351 and 2276 hr*mg/ml, respectively; Table 8). This observation supports the similar trends in weight changes in these two groups.

TABLE 8
PK study results

| | DTX | | C₀ | | | | AUCₗₐₛₜ |
|---|---|---|---|---|---|---|---|
| | dose | T_{1/2} | (ng/ | Tₗₐₛₜ | Cₘₐₓ | Tₘₐₓ | (hr * |
| Group | (mg/kg) | (hr) | ml) | (hr) | (ng/ml) | (hr) | mg/ml) |
| Formulation | 13 | 61.6 | 4 | 168 | 80.4 | 4 | 3345 |
| VI | | | | | | | |
| Formulation | 8.8 | 49.2 | 2 | 120 | 67 | 2 | 2351 |
| I | | | | | | | |
| i.v | 10 | 23.1 | 881 | 72 | 881 | 0 | 2276 |

Conclusions

Comparison between the systemic PK profile of docetaxel from the extended-release formulations (Formulations I and VI) and the i.v administration of docetaxel in rats demonstrated differences in the overall exposure time and the peak exposure. The overall exposure duration was longer in the extended-release formulations (both DTX concentrations) than the single i.v administration. The peak plasma level was higher following i.v administration of docetaxel. These differences are due to the slow and gradual release of docetaxel from the extended-release formulations. While Formulations I and VI extended the period of exposure by gradually releasing docetaxel, it also reduced the peak plasma level, limiting the potential exposure to cytotoxic concentrations. Tₗₐₛₜ increased with the dose of docetaxel in the extended-release formulations. The same relationship was observed for the AUC, Cₘₐₓ and t_{1/2}. This study demonstrated the that the prolonged release formulation according to exemplary embodiments of the invention release docetaxel over a prolonged period, while preserving systemic exposure (AUC) similar to the exposure of i.v treatment but with a greatly reduced Cₘₐₓ.

### Example 12—Evaluation of Local Safety of the Sustained Release Formulations According to Exemplary Embodiments of the Invention after Intracranial (IC) Administration in SD Rats

This study was performed to asses the local and the systemic safety of different amounts of the extended release formulation according to exemplary embodiments of the invention after IC administration in Sprague-Dawley rats.

Study design—Animals were divided into 7 groups (n=20/group). At study initiation the calvarium bone of the animals was exposed and 5 mm diameter defect was drilled in the calvarium bone, exposing the brain. In groups 1-3 Formulation II (50 mg, 25 mg and 10 mg; corresponding to 0.435, 0.218, and 0.087 mg of docetaxel, respectively further corresponding to 255 mg/cm², 127 mg/cm² and 51 mg/cm² of Formulation II (calculated based on a defect size of diameter of 0.5 cm having a surface area of 0.196 cm²) were administered on the animal brain. In groups 4-6 Formulation II vehicle (without docetaxel) (50 mg, 25 mg, and 10 mg) were administered on the animal brain. Group 7 served as sham control. Following test article administration, the defect was sealed with bone wax and the incision was sutured. Animals were returned to their cages to recover. Clinical signs, body weight and evaluation of the cognitive behavior (motility, tremor, head tilt and hair rotation) were followed during the study. At each designated time point (1, 4, 8 or 16 weeks), 5 animals from each group were sacrificed followed by gross necropsy and collection of administration site and vital organs for histopathology evaluation in a blind manner.

During the study only one animal (from the 25 mg treated group) was found dead on day 89. One animal (from the 50 mg treated group) was terminated early on day 90 due to severe weight loss. Both animals showed behavior changes scored as mild to moderate several days before the early termination or the death of the animal. The animal that was found dead did not undergo gross necropsy or histological evaluation because the long period of time that passed from the death until the time that it was found (˜24 hours). Gross necropsy and histological evaluation of the pre-terminated animal did not reveal any correlation between Formulation II administration and the animal situation, it was therefore concluded that weight loss was not related to the test article.

Aside from the single animal that suffered from severe weight loss (above), all other animals in all groups gained weight during the study.

Histopathology analysis of the skull and the brain from animals that were sacrificed one week after formulation II administration showed that similar average grades of inflammation (1.4-2.4) and necrosis (1.2-3.2) in the skull and in the cortex were present in all animals in all groups. No differences in the average scores were seen between the different doses of Formulation II and Formulation II vehicle (without docetaxel) in the treated groups.

Four weeks post administration, the average necrosis and inflammation scores in the skull and in the cortex decreased relative to the Week 1 scores in the sham and all Formulation II vehicle groups. In the animals from the groups treated with 50 and 25 mg Formulation II at Week 4, the average necrosis and inflammation scores generally increased relative to the Week 1 termination point. Scores in the 10 mg Formulation II group stayed constant between Weeks 1 and 4.

At the Week 8 termination time point, the average score of necrosis in the skull and cortex relative to the Week 4 termination time point decreased in severity in groups that were administered with 25 and 50 mg of Formulation II. The score for cortex inflammation was mild to moderate. In all other groups the score for inflammation and necrosis was none to minimal.

At the Week 16 termination time point, the average scores for necrosis and inflammation in all Formulation II treated groups were minimal, except the 25 mg treated group where the score was minimal-mild for necrosis in the skull. In the sham and Formulation II vehicle treated groups, the necrosis score was none and the inflammation score was minimal.

Conclusions

The administration of Formulation II did not cause any visible systemic adverse effects. The overall dose of docetaxel administered in Formulation II (i.e., up to 50 mg Formulation II, equivalent to 1-2 mg/kg docetaxel) is lower than the maximal tolerated dose (MTD) and non-lethal dose (NLD) reported (Taxotere (10 mg/kg iv); NDA 020449) as well to Docetaxel (NDA 205924).

Local release of cytotoxic drug caused local adverse effects; however these effects were resolved with time. This study supports the safety of administration of Formulation II up to overall dose of 50 mg/19.6 mm² in rat.

### Example 13—Evaluation of the Antitumor and Anti-Metastatic Effect of the Sustained Release Formulations According to Exemplary Embodiments of the Invention on LLC1 Cell Line In-Vivo Mouse Syngeneic Tumor Model

The objective of this study was to assess the efficacy of the antitumor and anti-metastatic effect of different amounts of Formulation II on mouse syngeneic louis lung carcinoma (LLC1) cell line tumors in C57BL mice. The selected cell line (LLC1) is known to spontaneously form metastases in the lungs originating from a primary tumor.

For that, subcutaneous colon carcinoma tumors were established in female BALB/c mice (7-8 weeks old, weighing ±16-20 gram at study initiation) and after reaching a desired volume (400-600 mm³), they were resected and ˜90% of their volume was removed followed by administration of the test items. The recurrence rate of the tumors was followed and compared to an untreated control group.

Male C57BL mice, 7-8 weeks old, weighing 18-21 grams were designated for this study. LLC1 tumor cells were injected SC to the back of the mice. When the tumors reached a volume of about 400 mm³ they were resected (at least 90% of the tumor volume was removed; average area of 0.7 cm²). The animals were divided into 6 groups (n=10). Study design details are listed in Table 9. Groups 1-4 were administered different amounts of Formulation II directly to the tumor bed. Untreated group (Group 5) served as negative control and systemically treated group (Group 6) served as positive control. Five (5) animals served as sham group, they were not injected with tumor cells but underwent the surgical procedure (Group 7). Following treatment, the surgical site was sewed and animals were returned to their cages for recovery. Animals that had tumors larger than 1500 mm³ were humanly terminated. At termination the number of metastases in the lungs was counted in each animal.

TABLE 9
Study design

| | | | | % Docetaxel | |
|---|---|---|---|---|---|
| | | | | (w/w) of the | Docetaxel |
| | | | | total weight | amount |
| | | | treatment | of the | (mg/ |
| Group | mice | test item | mg/animal | formulation | mouse) |
| 1 | 10 | Formulation II | 100 | 0.87 | 0.87 |
| 2 | 10 | Formulation II | 50 | 0.87 | 0.435 |
| 3 | 10 | Formulation II | 20 | 0.87 | 0.174 |
| 4 | 10 | Formulation II | 100 | NA | NA |
| | | Placebo | | | |
| 5 | 10 | Untreated | Saline | NA | NA |
| 6 | 10 | Positive control | 6 mg/kg IP | | |
| | | taxel | twice a week | | |
| 7 | 5 | Sham | NA | NA | NA |

Study Results

In Group 1 only one animal was early terminated on day 21. Although the tumor did not reach the maximal volume defined for early termination, the animal was sacrificed to verify if metastases developed in tumor bearing animals in this group. In Group 2 one animal was found dead on day 14. Three animals were early terminated, one on day 18 and two on day 21. One of the animals was terminated on day 21 to verify if metastases developed in tumor bearing animals in this group, although the tumor did not reach the maximal volume defined to early termination. The second was terminated due to its tumor size. In Group 3 four animals were found dead (on days 11, 18, and two on day 23). Four animals were early terminated on day 21 due to their tumor size. In Group 4, six animals were found dead (on days 14, 16, 3 on day 21 and 23). Two animals were early terminated on day 23 due to tumor that exceeded the maximal volume value determined for early termination. In Group 5 (untreated) one animal was found dead on day 16. Six animals were early terminated due to tumor size, three on day 14, two on day 16 and one on day 23. In Group 6, one animal was found dead on day 25. One animal was early terminated on day 23 due to tumor that exceeded the maximal volume value determined for early termination.

In all groups minor changes in the average body weight (%) were recorded. These changes were generally minimal (˜3%) and were most noted in Group 5 (untreated) and Group 6 (taxel), where the average weight at termination was 6.5% and 4% lower than their weight at t=0, respectively.

In Group 1, 6/10 animals had tumor with average tumor volume of 150 mm³. In Group 2, 8/10 animals had tumor with average tumor volume of 1363 mm³. In Group 3, 9/10 had tumor with average tumor volume of 2097 mm³. In Group 4, 6/10 had tumor with average tumor volume of 1559 mm³. In Group 5 (untreated), 7/10 had tumor with average tumor volume of 2463 mm³. In Group 6, 4/10 had tumor with average tumor volume of 490 mm³.

The number of metastases was counted post termination/death. In some cases, the lungs condition didn't allow evaluation of metastases. were too decomposed and therefore the number of metastases in these lungs were not evaluated. The counting discriminated between small metastases (0.1-0.5 mm) and big metastases (>0.5 mm). In case of large number of metastases (>100), it was defined as too numerus to count (TNTC).

In Group 1, 5/10 animals were metastases free. Three animals had small (0.1-0.5 mm) metastases (2, 6 and 7 metastases) and in the other two animals the lungs were too decomposed, and counting was impossible. The average lung weight was 198±55 mg. In Group 2, 4/10 animals were metastases free. Five animals had metastases. Two animals had small metastases (3 and 5 metastases), one animal had both small (0.1-0.5 mm) and large (>0.5 mm) metastases (11 and 6, respectively) and two animals had too numerus to count (TNTC) metastases (>100). In one animal the lungs were too decomposed for counting. The average lung weight was 252±87 mg. In Group 3, 3/10 animals were metastases free. Three animals had small metastases (5, 5 and 4 metastases), Three animals had both small and large metastases (6, 10 and 22 small; 1, 4 and 4 large, respectively) and one animal had TNTC metastases. The average lung weight was 323±115 mg. In Group 4, 2/10 animals were metastases free. Five animals had metastases. Three animals had small metastases (4, 7 and 9 metastases) and two animals had TNTC metastases. In three (3) animals the lungs were too decomposed for counting. The average lung weight was 587±481 mg. In Group 5 (untreated), all animals had metastases. Eight (8) animals had small metastases (varied between 2 to 20), one animal had both small and large metastases (5 and 3, respectively) and one animal had TNTC metastases. The average lung weight was 330±64 mg. In Group 6, 5/10 animals were metastases free. Four animals had metastases. Two animals had small metastases (3 and 4 metastases), one 1 animal had both small and large metastases (7 and 2, respectively) and one animal had TNTC metastases. In one animal the lungs were too decomposed for counting. The average lung weight was 226±114 mg.

Conclusions: In this study, treatment efficacy was evaluated by following the tumor volume and the number of metastases in the lungs following primary tumor surgical resection. The results of the study show that administration of a dose of 100 mg of Formulation II was effective in preventing tumor recurrence after tumor surgical resection, as well as preventing tumor cells from migrating, thus reducing the number of animal bearing metastases and the overall number of metastases in the lungs. These results pointing to the advantage of local treatment with the pharmaceutical compositions according to embodiments of the invention at the tumor bed for prevention of both tumor reoccurrence and metastases.

### Example 14—Evaluation of the Penetration of Taxane Released from a Pharmaceutical Composition According to Some Embodiments of the Invention into Rats Brain

Taxane sustained release composition according to certain embodiments of the invention (e.g. Formulation II) is administered into a 5-mm hole in the right hemisphere of a rats' brain. At different time points, a group of animals treated with the taxane sustained release composition will be sacrificed and their brain removed and analyzed for the presence of taxane. Specifically, the collected brains will be cut horizontally and vertically to form a 2 mm² cubes starting from the site of formulation II administration. The amount of docetaxel in each of the sliced cubes is determined using a validated Bioanalytical method for docetaxel in a rat brain tissue. The percentage of brain exposed to docetaxel, the diameter of the region exposed to the drug and the average concentration of the drug within this region are determined.

Methodology—

A 5-mm hole (19.6 mm²) is drilled deep through the middle of the calvarial bone above the right hemisphere using a trephine burr with constant saline irrigation to the level of the dura. Extreme care is taken to avoid damaging the dura matter. An elevator blade is placed into the defect margin and moved circumferentially around the defect until the drilled calvarium piece is raised and removed. The dura is then cut exposing the brain. Formulation II formulated as a paste is then applied on the brain surface.

All the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit, and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1-109. (canceled)

110. A method of treating a solid tumor comprising
administering to a subject with a solid tumor a pharmaceutical composition comprising:
a particulate biodegradable substrate coated or impregnated by a matrix composition comprising;
(a) a biodegradable polymer;
(b) at least one phospholipid having hydrocarbon chains of at least 12 carbons; and
(c) a taxane.

111. The method of claim 110, wherein the solid tumor is selected from the group consisting of: colon carcinoma, prostate cancer, pancreatic cancer, breast cancer, esophageal cancer, gastric cancer, head and neck cancer, and soft tissue sarcomas.

112. The method of claim 110, wherein the solid tumor is a brain tumor selected from a primary brain tumor or a metastatic brain tumor.

113. The method of claim 112, wherein the primary brain tumor is glioblastoma multiforme.

114. The method of claim 110, wherein the pharmaceutical composition is applied to the inner surface of a resection cavity of the solid tumor at a dose ranging from 20 mg to 260 mg per surface area of 1 cm².

115. The method of claim 110, wherein the taxane is selected from the group consisting of:
docetaxel, paclitaxel, and cabazitaxel.

116. The method of claim 115, wherein the taxane is docetaxel.

117. The method of claim 110, wherein the biodegradable polymer is a polyester.

118. The method of claim 110, wherein the at least one phospholipid is a phosphatidylcholine selected from the group consisting of: DMPC, DPPC, DSPC, and DOPC.

119. The method of claim 110, wherein the particulate biodegradable substrate comprises particles having an average particle size of less than about 200 μm.

120. The method of claim 110, wherein the particulate substrate comprises tri calcium phosphate.

121. The method of claim 110, wherein the particulate biodegradable substrate constitutes between about 80 to 93% (w/w) of the total weight of the pharmaceutical composition;
wherein the polymer constitutes between about 0.5-5% (w/w) of the total weight of the pharmaceutical composition; and
wherein the at least one phospholipid having hydrocarbon chains of at least 12 carbons constitutes between about 4.0-15% (w/w) of the total weight of the pharmaceutical composition.

122. The method of claim 110, wherein the taxane constitutes up to 2.6% (w/w) of the total weight of the pharmaceutical composition.

123. The method of claim 122, wherein the taxane constitutes between about 0.5 to 1.5% (w/w) of the total weight of the pharmaceutical composition.

124. The method of claim 110, wherein the pharmaceutical composition further comprises cholesterol.

125. The method of claim 110, further comprising a pH adjustment agent.

126. The method of claim 125, wherein the pharmaceutical composition comprises a pH between 4 and 6.

127. The method of claim 110, wherein the taxane penetrates to a distance of at least 1 cm away from the surface of the solid tumor,
wherein the solid tumor has been resected.

128. A method for treating a solid tumor, comprising:
administering to a subject with a solid tumor, a pharmaceutical composition comprising:
(a) 80%-93% (w/w) of β-tricalcium phosphate particles;
(b) 1%-4% (w/w) of PLGA;
(c) 4%-15% (w/w) DMPC;
(d) 0%-2% (w/w) of cholesterol; and
(e) 0.2% to 2.6% (w/w) of docetaxel.

129. The method of claim 110, wherein the solid tumor is a docetaxel resistant tumor.

130. The method of claim 110, wherein the pharmaceutical composition is in the form selected from the group consisting of: a powder, a paste; and a suspension for injection.
